SEQUENCE LISTING (5 sequences)

SEQ ID NO: 1 - DNA, 21 bp, Artificial Sequence
  chemically synthesized
cgccgtcctttgaataacaat

SEQ ID NO: 2 - DNA, 22 bp, Artificial Sequence
  chemically synthesized
agagggacatccaggactttgt

SEQ ID NO: 3 - DNA, 19 bp, Artificial Sequence
  chemically synthesized
caggcgcttgttggtgtac

SEQ ID NO: 4 - DNA, 20 bp, Artificial Sequence
  chemically synthesized
gcttgccctgtccagttaat

SEQ ID NO: 5 - DNA, 20 bp, Artificial Sequence
  chemically synthesized
tagctcagctgcacccttta

(19) United States
(12) Patent Application Publication
(10) Pub. No.: US 2015/0080390 A1
(43) Pub. Date: Mar. 19, 2015

(54) COMPOSITIONS USEFUL FOR TREATING HERPES SIMPLEX KERATITIS, AND METHODS USING SAME

(71) Applicant: Drexel University, Philadelphia, PA (US)

(72) Inventors: Jane E. Clifford, Narberth, PA (US); Oleg Alekseev, Philadelphia, PA (US); Stephen R. Jennings, Exton, PA (US)

(21) Appl. No.: 14/488,980

(22) Filed: Sep. 17, 2014

Related U.S. Application Data
(60) Provisional application No. 61/879,975, filed on Sep. 19, 2013.

(51) Int. Cl. A61K 31/5377; A61K 31/522

(52) U.S. Cl. CPC A61K 31/5377; A61K 31/522
USPC 514/232.8

(57) ABSTRACT

The present invention relates generally to compositions and methods for treating diseases and disorders caused by herpes simplex virus type 1, including herpes simplex keratitis, in a subject. In certain embodiments, the compositions of the present invention comprise an ATM inhibitor and an anti-herpetic agent. In other embodiments, the compositions comprise a Chk2 inhibitor and an anti-herpetic agent. In yet other embodiments, the compositions comprise a Chk2 inhibitor and an ATM inhibitor, and optionally an anti-herpetic agent.

### CROSS-REFERENCE TO RELATED APPLICATIONS

The present application claims priority under 35 U.S.C. §119(e) to U.S. Provisional Patent Application No. 61/879,975, filed Sep. 19, 2013, which is incorporated herein by reference in its entirety.

### STATEMENT REGARDING FEDERALLY SPONSORED RESEARCH OR DEVELOPMENT

This invention was made with government support under DK094612-01A1 awarded by National Institutes of Health. The government has certain rights in the invention.

### BACKGROUND OF THE INVENTION

Herpes simplex virus type 1 (HSV-1) is a ubiquitous pathogen capable of causing a range of ocular pathologies in the cornea, conjunctiva, uvea, and retina. HSV-1 invasion of the corneal epithelium results in a classical pattern of infection: the initial punctate lesions in the epithelium coalesce to form a dendritic ulcer, which expands further to become a geographic ulcer. If left untreated, herpetic ulcers may lead to permanent corneal scarring, thinning, opacification and neovascularization, with loss of vision, leaving corneal transplantation as the only option for restoration of sight, but with the risk of reactivated latent infection affecting the transplanted cornea. Herpes keratitis is the leading cause of both cornea-derived and infection-associated blindness in the developed world: about 500,000 cases in the U.S., with the annual incidence estimated at 11.8 per 100,000 people.

Clinical management of HSV infections largely relies on the use of nucleoside analogue antiviral drugs. In the U.S., HSV-1 keratitis is typically treated with topical ganciclovir, trifluridine, or vidarabine, as well as oral acyclovir. Topical corticosteroids are used to limit immune involvement in advanced cases of stromal keratitis, but can have the dangerous side effect of corneal melting or potentiate more severe infection. All of the current antiviral drugs exhibit varying degrees of corneal toxicity, which can become severe in prolonged treatments. This complicates the clinical management of difficult and refractory cases.

The emergence of drug-resistant HSV-1 strains is an additional concern. Wide use of acyclovir for the treatment of herpetic infections has resulted in many reports of clinically isolated resistant strains. Drug resistance is particularly high in the immunocompromised population, since the immune system normally promotes HSV-1 latency in the trigeminal ganglion and is instrumental in clearing the epithelial disease. Two main resistance mechanisms are known—at the thymidine kinase (TK) stage and at the DNA polymerase stage. Resistance through mutation of the TK gene is seen for drugs that require activation by the viral TK (e.g., acyclovir, ganciclovir, idoxuridine), but some resistant DNA polymerase mutants have also been reported. Cross-resistance between nucleoside analogue drugs further complicates the problem, highlighting the need for development of novel antiviral therapies.

HSV-1 interacts with host molecular machinery to optimize various aspects of the cellular environment for its own replication. The virus controls fundamental cellular functions, such as transcription, translation, cell cycle, autophagy, apoptosis, nuclear architecture, and antigen presentation. Among the host pathways hijacked by HSV-1 is the DNA damage response (DDR), which is a complex network of proteins responsible for the maintenance of genomic integrity of the cell. Sensor proteins of the DDR respond to DNA lesions and promote their repair by facilitating the assembly of repair proteins at the damaged DNA loci. Simultaneously, the DDR induces temporary cell cycle arrest to prevent the lesion from being passed on to the daughter cells. The DDR also induces transcriptional changes to optimize the cellular response to the incurred lesion. In the case of overwhelming or irreparable damage, the DDR promotes apoptosis of the affected cell. Three main sensor kinases serve as the apical proteins in the DDR: ATM (ataxia telangiectasia mutated), ATR (ataxia telangiectasia and Rad3 related), and DNA-PK (DNA-dependent protein kinase). There are no reported studies of the relationship between HSV-1 and the DDR specifically in the corneal epithelium.

Therefore, there is thus a need in the art for improved compositions and methods for the treatment of HSV. The present invention satisfies this unmet need.

### BRIEF SUMMARY OF THE INVENTION

In one aspect, the invention provides a composition comprising an anti-herpetic agent and at least one inhibitor selected from the group consisting of an ATM inhibitor, a Chk2 inhibitor, and a salt, solvate or N-oxide thereof, wherein the composition treats or prevents herpes simplex keratitis in a subject in need thereof. In another aspect, the invention provides a method of treating or preventing herpes simplex keratitis in a subject in need thereof. In yet another aspect, the invention provides a method of treating or preventing herpes simplex keratitis in a subject in need thereof, wherein the keratitis is caused by a drug-resistant HSV-1 strain. In yet another aspect, the invention provides a kit comprising at least one inhibitor selected from the group consisting of an ATM inhibitor and a Chk2 inhibitor, the kit further comprising an applicator; and an instructional material for the use of the kit, wherein the instruction material comprises instructions for treating, ameliorating or preventing herpes simplex keratitis in a subject in need thereof.

In certain embodiments, the ATM inhibitor is at least one selected from the group consisting of a nucleic acid, siRNA, antisense nucleic acid, ribozyme, peptide, small molecule, antagonist, aptamer, and peptidomimetic. In other embodiments, the small molecule is at least one selected from the group consisting of caffeine, wortmannin, chloroquine, CP-466722, KU-55933, KU-59403, KU-60019, and a salt, N-oxide or solvate thereof.

In certain embodiments, the Chk2 inhibitor is at least one selected from the group consisting of a nucleic acid, siRNA, antisense nucleic acid, ribozyme, peptide, small molecule, antagonist, aptamer, and peptidomimetic. In other embodiments, the small molecule is at least one selected from the group consisting of Chk2 inhibitor II, SC-203885, NSC-109555, and a salt, N-oxide or solvate thereof.

In certain embodiments, the anti-herpetic agent is at least one selected from the group consisting of acyclovir, famciclovir, penciclovir, valacyclovir, acyclovir, trifluridine, penciclovir and valacyclovir.

In certain embodiments, the method of the present invention comprises administering to the subject an effective amount of an anti-herpetic agent and an effective amount of at least one inhibitor selected from the group consisting of an ATM inhibitor and a Chk2 inhibitor, whereby herpes simplex keratitis is treated or prevented in the subject.

In certain embodiments, the method of the present invention comprises administering to the subject an effective amount of at least one inhibitor selected from the group consisting of an ATM inhibitor and a Chk2 inhibitor, wherein the subject is optionally further administered an effective amount of an anti-herpetic agent, whereby herpes simplex keratitis is treated or prevented in the subject.

In certain embodiments, the at least one inhibitor and the anti-herpetic agent are co-administered to the subject. In other embodiments, the at least one inhibitor and the anti-herpetic agent are co-formulated. In yet other embodiments, the inhibitor is administered to the subject by a topical or intraocular route.

In certain embodiments, administration of the inhibitor to the subject reduces the amount of the anti-herpetic agent required to be administered to the subject to obtain the same therapeutic benefit obtained when the effective dose of the anti-herpetic agent in the absence of the inhibitor is administered to the subject.

In certain embodiments, the subject experiences less frequent or less severe side effects of the anti-herpetic agent, as compared to when the effective dose of the anti-herpetic agent in the absence of the inhibitor is administered to the subject.

In certain embodiments, development of resistance to the anti-herpetic agent is prevented or minimized in the subject, as compared to when the effective dose of the anti-herpetic agent in the absence of the inhibitor is administered to the subject.

In certain embodiments, the subject is a mammal. In other embodiments, the mammal is a human.

In certain embodiments, the drug-resistant HSV-1 strain has a TK mutation. In other embodiments, the strain is resistant to at least one selected from the group consisting of acyclovir, famciclovir, penciclovir, valacyclovir, acyclovir, trifluridine, penciclovir and valacyclovir.

In certain embodiments, the kit further comprises an anti-herpetic agent.

### BRIEF DESCRIPTION OF THE DRAWINGS

The following detailed description of specific embodiments of the present invention will be better understood when read in conjunction with the appended drawings. For the purpose of illustrating the invention, specific embodiments are shown in the drawings. It should be understood, however, that the invention is not limited to the precise arrangements and instrumentalities of the embodiments shown in the drawings.

FIGS. 1A-1C illustrate the finding that HSV-1 activates ATM in human corneal epithelial cells. FIG. 1A: hTCEpi cells were infected with HSV-1 at MOI 5.0. Lysates were collected at the indicated time points and analyzed by Western blot with antibodies specific to the indicated proteins. ICP0 staining was used to mark the progression of infection, and nucleolin is a loading control. Thr68 is an ATM-specific phosphorylation site on Chk2. FIG. 1B: hTCEpi cells were infected with HSV-1 at MOI 5.0 and fixed at the indicated hpi. Cells were processed for indirect immunofluorescence with the indicated primary antibodies and counterstained with Hoechst 33258. ICP8 staining was used to visualize the viral replication compartments. Scale Bar: 10 μm. Data are representative of at least three independent experiments. FIG. 1C: set of images illustrating the results of experiments where HSV-1 infected hTCEpi cells were fixed 8 hours post infection and stained for the presence of activated ATM or Chk2.

FIGS. 2A-2F illustrate the finding that ATM inhibition suppresses HSV-1 replication in vitro. hTCEpi cells were infected at MOI 0.1 in the presence of ATM inhibitor (KU-55933, 10 μM). Control cells were neither infected nor treated. Mock treatment (DMSO) and viral replication inhibitor (PAA, 400 μg/mL) were used as negative and positive treatment controls, respectively. Under these experimental conditions, PAA contains activities that were found to inhibit several stages of HSV-1 gene expression. FIG. 2A: Phase contrast images of hTCEpi cells were taken at 20 hpi. FIG. 2B: Supernatants were collected at the indicated time points for analysis by plaque assay. Bars represent average viral titers±SEM. FIG. 2C: Total DNA was collected at the indicated time points for analysis by qPCR with primers for HSV-1 DNA polymerase and GAPDH. A representative experiment is shown. FIG. 2D: Results of a plaque assay using HSV-1 infected hTCEpi cells. FIG. 2E: Results of a plaque assay using HSV-1 infected HCE cells. FIG. 2F: Number of HSV genome copies in HSV-1 infected HCE cells. Bars represent relative ΔΔC(t) values±SEM. n=3 for all.

FIGS. 3A-3B illustrate the finding that ATM inhibition reduces accumulation of viral transcripts and proteins in vitro. hTCEpi cells were infected at MOI 0.1 in the presence of ATM inhibitor (KU-55933, 10 μM). Mock treatment (DMSO) and viral replication inhibitor (PAA, 400 μg/mL) were used as negative and positive controls, respectively. Under these experimental conditions, PAA contains activities that were found to inhibit several stages of HSV-1 gene expression. Cells were collected for protein lysates or RNA isolation at 16 hpi. FIG. 3A: Transcripts from all three HSV-1 gene families were detected with primers for ICP0 (immediate early), DNA polymerase (early), and glycoprotein C (true late). Bars represent relative ΔΔC(t) values±SEM. FIG. 3B: Viral protein accumulation was assayed by Western blot with antibodies against ICP0 and ICP4 (immediate early), ICP8 (early), glycoprotein B (leaky late), and glycoprotein C (true late). Control lysates were collected from cells that were neither infected nor treated. Nucleolin is a loading control. n=2 for all.

FIGS. 4A-4E illustrate the finding that ATM inhibition suppresses HSV-1 replication in explanted human and rabbit corneas. FIG. 4A: Schematic representation of the ex vivo culture method of explanted corneoscleral buttons. FIG. 4B: Ex vivo human corneas were pretreated for 1 hour with ATM inhibitor (KU-55933, 10 μM) or DMSO, followed by administration of bleomycin (200 μg/mL) for an additional hour. The epithelial layers were collected for protein lysates and analyzed by Western blot with antibodies against pATM (Ser1981) and total ATM. Each lysate was collected from three pooled corneas. FIGS. 4C-4D: Human and rabbit corneas were infected with 13104 PFU/cornea. Treatments were applied at 1 hpi: ATM inhibitor (KU-55933, 10 μM) and mock treatment (DMSO). FIG. 4C: PAA (400 μg/mL) was included as a positive control. Under these experimental conditions, PAA contains activities that were found to inhibit several stages of HSV-1 gene expression. DNA was isolated from the epithelial layers at 48 hpi and analyzed by qPCR with primers for HSV-1 DNA polymerase and GAPDH. Bars represent relative ΔΔC(t) values±SEM. n=6 for each treatment. FIG. 4D: Human corneas were processed for indirect immunofluorescence staining for cleaved caspase-3. Counterstain is Hoechst 33258. FIG. 4E: Fold change in Pol transcript in untreated and treated infected corneas. n=3.

FIGS. 5A-5C illustrate the finding that KU-55933 reduces disease severity in the mouse model of herpes keratitis. FIG. 5A: Corneas of 3-week-old C57BL/6J mice were infected with McKrae strain of HSV-1. Treatments with 200 μM KU-55933 (represented by black dots in the schematic) were initiated at 24 hpi and administered every 4 hours for 1 full day and then every 8 hours for the remainder of the experiment. dpi=days postinfection. Ocular disease severity was scored on a number scale for stromal keratitis (FIG. 5B) and blepharitis (FIG. 5C). Data points represent average disease scores±SEM. n=5 mice per group.

FIGS. 6A-6C illustrate the finding that KU-55933 exhibits low toxicity in corneal epithelium. FIG. 6A: The toxicity of ATM inhibition in hTCEpi cells was assessed by colony survival assay after a 24-hour treatment with KU-55933 (10 μM). Bars represent average colony survival±SEM. n=3. FIG. 6B: Ex vivo human corneas were treated with KU-55933 (10 μM) continually for 30 hours, and the epithelial toxicity was assessed by fluorescein staining. Toxic treatment with doxorubicin (100 μM) for 30 hours served as a positive control for detection of damage by staining. n=2. FIG. 6C: The eyes of uninfected healthy mice were treated with 200 μM KU-55933 administered at the same frequency and duration (4 days) as in the mouse ocular infection experiments (FIG. 5A). At the end of the experiment, the treated corneas were assessed for toxicity by fluorescein staining. A mouse cornea de-epithelialized as a consequence of untreated HSV-1 infection served as a positive staining control. n=2.

FIGS. 7A-7B illustrate the finding that ATM inhibition enhances the antiviral activity of acyclovir. hTCEpi cells were infected at MOI 0.1 in the presence of 16 different dose combinations of KU-55933 (0, 2, 4, and 7 μM) and acyclovir (0, 0.2, 0.5, and 1.5 μg/mL). Total DNA was collected at 16 hpi for analysis by qPCR with primers for HSV-1 polymerase and GAPDH. Viral genome replication was calculated using the ΔΔC(t) method. Data are representative of at least two independent experiments. The same data set was plotted in two different ways to highlight (FIG. 7A) the effect of KU-55933 on the acyclovir dose-response curve and (FIG. 7B) the effect of acyclovir on the KU-55933 dose-response curve.

FIG. 8 is a graph that illustrates the finding that ATM inhibition suppresses acyclovir-resistant HSV-1 infection. hTCEpi cells were infected at MOI 0.1 with wild-type or acyclovir-resistant HSV-1 (KOS strain and dlsptk strain, respectively) in the presence of ATM inhibitor (KU-55933, 10 μM). Mock treatment (DMSO) and viral polymerase inhibitor (acyclovir, 50 μg/mL) were used as negative and positive controls, respectively. Total DNA was collected at 16 hpi for analysis by qPCR with primers for HSV-1 polymerase and GAPDH. All values are normalized to the corresponding DMSO samples. Bars represent relative ΔΔC(t) values±SEM. n=2.

FIGS. 9A-9B are a set of schematics illustrating the role of ATM/Chk2 in the DNA damage response signaling cascade (FIG. 9A) and an overview of the HSV-1 life cycle in the context of HSK (FIG. 9B).

FIGS. 10A-10E illustrate the finding that inhibition of ATM or Chk2 blocks viral transcription. FIGS. 10A-10D are graphs depicting the levels of ICP0 (FIG. 10A), TK (FIG. 10B), gC (FIG. 10C), and latency-associated transcript (FIG. 10D) in treated and untreated infected hTCEpi cells. FIG. 10E is a graph depicting the level of Pol transcript in infected cells treated with ATM shRNA or control (scrambled shRNA). n=3. Error bars indicate±SEM.

FIGS. 11A-11B illustrate the finding that Chk2 inhibition suppresses HSV-1 cytopathic effect in human corneal epithelial cells. FIG. 11A: hTCEpi cells were infected with HSV-1 at MOI 5.0. Lysates were collected at the indicated time points and analyzed by Western blot with antibodies specific to the indicated proteins. pATM antibody detects autophosphorylation of ATM on Ser 1981, and pChk2 antibody detects its activation by phosphorylation on Thr 68 by ATM. Nucleolin is a loading control. FIG. 11B: hTCEpi cells were infected at MOI 0.1 in the presence of Chk2 inhibitor II (10 μM). Control cells were neither infected nor treated. Mock treatment (DMSO) and viral polymerase inhibitor (PAA, 400 μg/ml) were used as negative and positive treatment controls, respectively. Phase contrast images were taken at 20 hpi. A representative field is shown for each treatment. n=at least 5 independent experiments. hpi=hours post infection.

FIGS. 12A-12B illustrate the finding that Chk2 inhibition suppresses HSV-1 genome replication in vitro. FIG. 12A: hTCEpi and (FIG. 12B) HCE cells were infected at MOI 0.1 in the presence of Chk2 inhibitor II (10 μM). Mock treatment (DMSO) and viral polymerase inhibitor (PAA, 400 μg/ml) were used as negative and positive treatment controls, respectively. Total DNA was collected at the indicated time points for analysis by qPCR with primers for HSV-1 polymerase and GAPDH. Values represent average ΔΔC(t)±SEM. n=3 experimental replicates.

FIGS. 13A-13B illustrate the finding that Chk2 inhibition suppresses HSV-1 infectious particle production in vitro. (FIG. 13A) hTCEpi and (FIG. 13B) HCE cells were infected at MOI 0.1 in the presence of Chk2 inhibitor II (10 μM). Mock treatment (DMSO) and viral polymerase inhibitor (PAA, 400 μg/ml) were used as negative and positive treatment controls, respectively. Supernatants were collected at the indicated time points for analysis by plaque assay. Values represent average viral titers±SEM for a representative of at least 3 independent experiments. n=3 plaque assay replicates.

FIG. 14 is a graph that illustrates the finding that Chk2 inhibition suppresses HSV-1 replication in vitro at a high viral load. hTCEpi cells were infected at MOI 5.0 in the presence of Chk2 inhibitor II (10 μM). Mock treatment (DMSO) and viral polymerase inhibitor (PAA, 400 μg/ml) were used as negative and positive treatment controls, respectively. Total DNA was collected at the indicated time points for analysis by qPCR with primers for HSV-1 polymerase and GAPDH. Values represent average ΔΔC(t)±SEM. n=3 experimental replicates.

FIG. 15 is a bar graph that illustrates the finding that Chk2 knockdown reduces HSV-1 replication in vitro. HCE cells harboring tetracycline-inducible expression of shRNA against Chk2 or non-targeting control were cultured in the presence of doxycycline (0.25 μg/ml) for 72 hours to induce Chk2 knockdown. Following the induction, cells were infected with HSV-1 at MOI 0.1, and total DNA was collected at the indicated time points for analysis by qPCR with primers for HSV-1 polymerase and GAPDH. Doxycycline was present in the medium for the entire duration of infection. Protein lysates were collected at the time of infection to verify knockdown by Western blot (inset). Nucleolin is a loading control. Values represent average ΔΔC(t)±SEM for a representative of two independent experiments. n=2 reaction replicates.

FIG. 16 is a bar graph that illustrates the finding that Chk2 inhibition suppresses HSV-1 replication in explanted human and rabbit corneas. Human and rabbit corneas were infected with 1×10⁴ PFU/cornea. At 1 hpi, they were treated with Chk2 inhibitor II (10 μM). Mock treatment (DMSO) and PAA (400 μg/ml) were included as negative and positive controls, respectively. DNA was isolated from the epithelial layers at 48 hpi and analyzed by qPCR with primers for HSV-1 DNA polymerase and GAPDH. Bars represent average ΔΔC(t) values±SEM. n=6 corneas per treatment.

FIG. 17 is a bar graph that illustrates the finding that the effect of Chk2 inhibition on HSV-1 replication in explanted corneas is prolonged. Rabbit corneas were infected with 1×10⁴ PFU/cornea and treated with Chk2 inhibitor II (10 μM) or mock treatment (DMSO) for 48 hours. Corneas were rinsed and cultured in fresh inhibitor-free medium for additional 48 hours (inset), during which time total DNA was isolated from the epithelial layers at the indicated time points () and analyzed by qPCR with primers for HSV-1 DNA polymerase and GAPDH. Bars represent average ΔΔC(t) values±SEM. n=6 corneas per each timepoint and treatment.

FIG. 18 is a set of images that illustrates the finding that Chk2 inhibition reduces HSV-1-associated apoptosis in explanted corneas. Ex vivo human corneas were infected with 1×10⁴ PFU/cornea and treated with Chk2 inhibitor II (10 μM) or mock treatment (DMSO). Corneas were flash-frozen at 48 hours and processed for indirect immunofluorescence staining with antibodies against cleaved caspase 3. Counterstain is Hochst 33258. A representative limbal field for each treatment is shown. n=2 corneas per treatment.

FIGS. 19A-19B illustrate the dose-optimization of Chk2 inhibitor II in human corneal epithelium. FIG. 19A: hTCEpi cells were infected with HSV-1 at MOI 0.1 and treated with a dose range (0-10 μM) of Chk2 inhibitor II. FIG. 19B: Human corneas were infected ex vivo with 1×10⁴ PFU/cornea. At 1 hpi, they were treated with a dose range (10-30 μM) of Chk2 inhibitor II. DNA was isolated from cultured cells and corneal epithelial layers at 20 hpi and 48 hpi, respectively, and analyzed by qPCR with primers for HSV-1 DNA polymerase and GAPDH. Bars represent average ΔΔC(t) values±SEM. n=3 reaction replicates.

FIGS. 20A-20D illustrate the finding that HSV-1 activates ATM in the absence of DNA damage. FIG. 20A: EPC2 cells were infected with HSV-1 at MOI 5, and protein lysates were analyzed by Western blot with the indicated antibodies. pATM-Ser1981, pChk2-Thr68. ICP0 staining marks the progress of infection; nucleolin is a loading control. hpi=hours post infection. FIG. 20B: Top panels: HEK293 cells were transfected with fHSVΔpac BAC, and hTCEpi cells were infected with HSV-1 at MOI 5. After 26 hours and 4 hours, respectively, cells were fixed and stained for pATM (Ser1981). Bottom panels: HEK293 cells were transfected with HSV-1 KOS genome, maintained in the absence or presence of PAA for 24 hours, and stained for pATM (Ser1981). ICP8 served as a marker of replication compartments. FIGS. 20C-20D: OKF6 cells were treated with 150 μM H₂O₂ for 1.5 hours or infected with HSV-1 at MOI 5 for 5 hours. FIG. 20C: Protein lysates were analyzed by Western blot with the indicated antibodies. A representative blot is shown, along with a quantification of pATM/tATM ratios from two independent experiments. FIG. 20D: Levels of DNA damage (single and double strand breaks) sustained by the cells were measured by comet assay. A representative set of comet images is shown, along with a quantification of Olive moment measurements (60 cells per treatment from two independent experiments). Bar=mean±SEM.

FIGS. 21A-21D illustrate the finding that ATM activation requires nuclear entry of the genome and is only partial in the absence of de novo protein synthesis. hTCEpi cells were infected with HSV-1 at a range of MOIs in the presence or absence of (FIG. 21A) PAA (400 μg/ml) or (FIG. 21B) CHX (5 μg/ml) with virus that had been exposed to UV (0.2 J/cm²) or mock treated prior to infection. FIG. 21C: Synchronized infection was set up in hTCEpi cells in the presence or absence of CHX (5 μg/ml). FIG. 21D: hTCEpi cells were infected with the tsB7 strain of HSV-1 at permissive (34° C.) or non-permissive (39° C.) temperature. For all experiments, protein lysates were collected at 1 hpi, except FIG. 21C, where lysates were collected at 10 min intervals for the first hour of infection. n≧2 independent experiments.

FIGS. 22A-22C illustrate the finding that HSV-1 activates ATM in an ICP4-dependent manner. FIG. 22A: Confluent monolayers of hTCEpi cells were infected with ICP0-null or WT HSV-1 at low MOI and overlaid with methocellulose-containing medium. Once plaques developed, cells were fixed and stained for pATM (Ser1981). ICP8 served as a marker of infected cells. FIG. 22B: hTCEpi cells were infected with ICP4-null or WT HSV-1 at a range of MOIs in the presence or absence of CHX (5 μg/ml). Protein lysates were analyzed by Western blot with the indicated antibodies. FIG. 22C: HEK293 cells were transfected with an ICP4-null HSV-1 BAC (pM24 BAC) or the complete purified HSV-1 KOS genome. Cells were fixed after 24 hours and stained for pATM (Ser1981). GFP fluorescence (BAC) and ICP8 staining (genome) were used as markers of transfected cells. n≧2 independent experiments.

FIGS. 23A-23B illustrate the finding that ATM activity is critical to HSV-1 replication at the onset of infection. hTCEpi cells were infected with HSV-1 at MOI 1, with KU-55933 (10 μM) treatments initiated at the indicated number of hours with respect to the time of infection (O). Protein lysates and total DNA were collected from cells at 8 hpi and analyzed by (FIG. 23A) Western blot with the indicated antibodies and (FIG. 23B) qRT-PCR with primers for the viral genome. GAPDH served as a reference gene. Raw data were processed by the ΔΔC(t) method. Bar=mean±SEM. n≧2 independent experiments.

FIG. 24 is a set of images illustrating immunofluorescence results.

FIGS. 25A-25B illustrate western blot results.

FIG. 26A is a graph illustrating the relative genome level for various cell lines treated with DMSo or KU-55933. FIG. 26B is a set of images illustrating western blot results.

### DETAILED DESCRIPTION OF THE INVENTION

The present invention relates generally to compositions and methods for treating diseases and disorders caused by herpes simplex virus type 1, including herpes simplex keratitis, in a subject. In one aspect, the present invention provides a composition for treating herpes simplex keratitis in a subject. In certain embodiments, the compositions of the present invention comprise an ATM inhibitor and an anti-herpetic agent. In other embodiments, the compositions comprise a Chk2 inhibitor and an anti-herpetic agent. In yet other embodiments, the compositions comprise a Chk2 inhibitor and an ATM inhibitor, and optionally an anti-herpetic agent.

In one aspect, the present invention provides a method of treating or preventing herpes simplex keratitis in a subject in need thereof. In certain embodiments, the method comprises administering to the subject an effective amount of a composition comprising an ATM inhibitor and an anti-herpetic agent. In other embodiments, the method comprises administering to the subject an effective amount of a composition comprising a Chk2 inhibitor and an anti-herpetic agent. In yet other embodiments, the method comprises administering to the subject an effective amount of a composition comprising an ATM inhibitor, a Chk2 inhibitor and optionally an anti-herpetic agent. In yet other embodiments, the method comprises administering to the subject an effective amount of an ATM inhibitor and an effective amount of an anti-herpetic agent. In yet other embodiments, the method comprises administering to the subject an effective amount of a Chk2 inhibitor and an effective amount of an anti-herpetic agent. In yet other embodiments, the method comprises administering to the subject an effective amount of a Chk2 inhibitor, an effective amount of an ATM inhibitor and optionally an effective amount of an anti-herpetic agent.

In certain embodiments, administration of an ATM inhibitor reduces the effective amount of the anti-herpetic agent required to be administered to the subject to obtain the same therapeutic benefit. In other embodiments, administration of a Chk2 inhibitor reduces the effective amount of the anti-herpetic agent required to be administered to the subject to obtain the same therapeutic benefit. In yet other embodiments, the reduced effective amount of the anti-herpetic agent required to be administered to the subject to obtain the same therapeutic benefit results in a reduced frequency or severity of side effects due to the anti-herpetic agent experienced by the subject. In yet other embodiments, the infection is caused by a drug-resistant HSV-1 strain. In yet other embodiments, the drug-resistant HSV-1 strain has a TK mutation. In yet other embodiments, the strain is resistant to at least one selected from the group consisting of acyclovir, famciclovir, penciclovir, valacyclovir, acyclovir, trifluridine, penciclovir and valacyclovir.

As demonstrated herein, ATM is a significant participant in HSV-1 infection of corneal epithelium. ATM is rapidly activated in response to infection, and inhibition of its kinase activity with a small molecule inhibitor, KU-55933,28 greatly reduces replication of the virus and the cytopathic effect produced in the infected cells. The antiviral activity of KU-55933 was demonstrated in the human and rabbit corneal explant models, as well as in the mouse model of ocular HSV-1 keratitis. In cultured cells, KU-55933 allowed for a lower dosage of co-administered acyclovir. Further, KU-55933 effectively suppressed replication of a drug-resistant HSV-1 strain harboring a TK mutation. The present results demonstrate that ATM is a therapeutic target for the treatment of HSV-1 keratitis.

As further demonstrated herein, Chk2 activation occurs very early in the course of HSV-1 infection, and inhibition of Chk2 kinase activity potently suppresses viral replication in human corneal epithelial cells, as well as in organotypically explanted human and rabbit corneas. The present work thus identifies Chk2 as a therapeutic target in the treatment of HSV-1 corneal infection.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

Generally, the nomenclature used herein and the laboratory procedures in cell culture, molecular genetics, organic chemistry, virology, and nucleic acid chemistry and hybridization are those well-known and commonly employed in the art. The nomenclature used herein and the laboratory procedures used in analytical chemistry described below are those well known and commonly employed in the art. Standard techniques or modifications thereof, are used for chemical syntheses and chemical analyses.

Standard techniques are used for nucleic acid and peptide synthesis. The techniques and procedures are generally performed according to conventional methods in the art and various general references (e.g., Sambrook and Russell, 2012, Molecular Cloning, A Laboratory Approach, Cold Spring Harbor Press, Cold Spring Harbor, N.Y., and Ausubel et al., 2002, Current Protocols in Molecular Biology, John Wiley & Sons, NY), which are provided throughout this document.

As used herein, each of the following terms has the meaning associated with it in this section.

As used herein, the articles “a” and “an” refer to one or to more than one (i.e., to at least one) of the grammatical object of the article. By way of example, “an element” means one element or more than one element.

As used herein, “about” when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, a disease or disorder is “alleviated” if the severity or frequency of at least one sign or symptom of the disease or disorder experienced by a patient is reduced.

As used herein, the term “analog” or “analogue” or “derivative” is meant to refer to a chemical compound or molecule made from a parent compound or molecule by one or more chemical reactions. As such, an analog can be a structure having a structure similar to that of the small molecule inhibitors described herein or can be based on a scaffold of a small molecule inhibitor described herein, but differing from it in respect to certain components or structural makeup, which may have a similar or opposite action metabolically. An analog or derivative of any of a small molecule inhibitor in accordance with the present invention can be used within the methods of the present invention.

As the term is used herein, “applicator” is used to identify any device including, but not limited to, a hypodermic syringe, pipette, nebulizer, vaporizer and the like, for administering the compounds and compositions used in the practice of the present invention.

As used herein, the term “ATM” kinase refers to ataxia telangiectasia mutated kinase.

As used herein, the term “ATR” kinase refers to ataxia telangiectasia and Rad3 related kinase.

As used herein, the phrase “ATM inhibitor” or “inhibitor of ATM” refers to a composition or compound that inhibits ATM activity, either directly or indirectly, using any method known to the skilled artisan. An ATM inhibitor may be any type of compound, including but not limited to, a nucleic acid, peptide, antibody, small molecule, antagonist, aptamer, or peptidomimetic.

As used herein, the phrase “Chk2 inhibitor” or “inhibitor of Chk2” refers to a composition or compound that inhibits Chk2 activity, either directly or indirectly, using any method known to the skilled artisan. A Chk2 inhibitor may be any type of compound, including but not limited to, a nucleic acid, peptide, antibody, small molecule, antagonist, aptamer, or peptidomimetic.

As used herein, the phrase “Chk2 inhibitor II” refers to 2-(4-(4-chlorophenoxy)phenyl)-1H-benzimidazole-5-carboxamide, or a salt, N-oxide or solvate thereof:

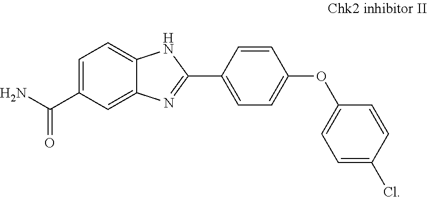 [SMILES: NC(=O)c1ccc2[nH]c(-c3ccc(Oc4ccc(Cl)cc4)cc3)nc2c1]

As used herein, the term “chloroquine” refers to N⁴-(7-chloro-4-quinolinyl)-N1,N1-diethyl-1,4-pentanediamine, or a salt, N-oxide or solvate thereof:

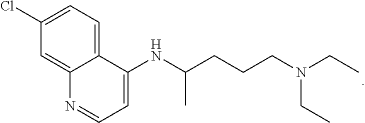 [SMILES: CCN(CC)CCCC(C)Nc1ccnc2cc(Cl)ccc12]

As used herein, the term “CP-466722” or “CP466722” refers to 2-(6,7-dimethoxyquinazolin-4-yl)-5-(pyridin-2-yl)-2H-1,2,4-triazol-3-amine, or a salt, N-oxide or solvate thereof:

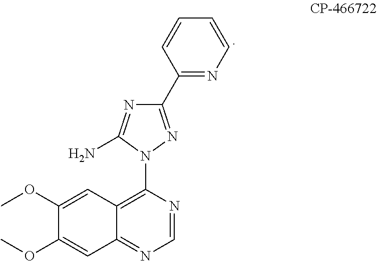 [SMILES: COc1cc2ncnc(-n3nc(-c4ccccn4)nc3N)c2cc1OC]

As used herein, the term “container” includes any receptacle for holding the pharmaceutical composition. For example, in certain embodiments, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well-known in the art. It should be understood that the instructions for use of the pharmaceutical composition may be contained on the packaging containing the pharmaceutical composition, and as such the instructions form an increased functional relationship to the packaged product. However, it should be understood that the instructions can contain information pertaining to the compound's ability to perform its intended function, e.g., treating, ameliorating, or preventing HSV-1 infection in a subject.

As used herein, the term “DDR” refers to DNA damage response.

As used herein, a “disease” is a state of health of an animal wherein the animal cannot maintain homeostasis, and wherein if the disease is not ameliorated then the animal's health continues to deteriorate.

As used herein, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “DNA-PK” refers to DNA-dependent protein kinase.

As used herein, the term “dpi” refers to days postinfection.

As used herein, the terms “effective amount” and “pharmaceutically effective amount” and “therapeutically effective amount” refer to an amount of an agent to provide the desired biological or therapeutic result. That result can be reduction and/or alleviation of the signs, symptoms, or causes of a disease or disorder, or any other desired alteration of a biological system. An appropriate effective amount in any individual case may be determined by one of ordinary skill in the art using routine experimentation.

As used herein, the term “endogenous” refers to any material from or produced inside an organism, cell, tissue or system.

As used herein, the term “exogenous” refers to any material introduced from or produced outside an organism, cell, tissue or system.

As used herein, the term “expression” is defined as the transcription and/or translation of a particular nucleotide sequence driven by its promoter.

As used herein, the term “HSV-1” refers to herpes simplex virus type 1.

As used herein, the terms “inhibit” and “inhibition” mean to reduce a molecule, a reaction, an interaction, a gene, an mRNA, and/or a protein's expression, stability, function or activity by a measurable amount or to prevent entirely. “Inhibitors” are compounds that, e.g., bind to, partially or totally block stimulation, decrease, prevent, delay activation, inactivate, desensitize, or down regulate a protein, a gene, and an mRNA stability, expression, function and activity, e.g., antagonists.

“Instructional material,” as that term is used herein, includes a publication, a recording, a diagram, or any other medium of expression which can be used to communicate the usefulness of a composition of the present invention in a kit. The instructional material of the kit may, for example, be affixed to a container that contains a composition of the present invention or be shipped together with a container which contains a composition. Alternatively, the instructional material may be shipped separately from the container with the intention that the recipient uses the instructional material and a composition cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

As used herein, the term “KU-55933” or “KU55933” refers to 2-(morpholin-4-yl)-6-(thianthren-1-yl)-pyran-4-one, or a solvate, salt, N-oxide, or prodrug thereof:

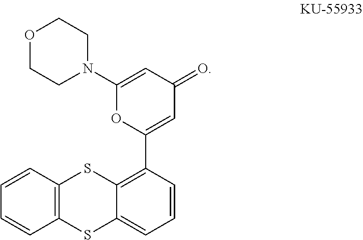 [SMILES: O=c1cc(-c2cccc3c2Sc2ccccc2S3)oc(N2CCOCC2)c1]

As used herein, the term “KU-59403” or “KU59403” refers to 3-(4-methyl piperazin-1-yl)-N-(6-(6-morpholino-4-oxo-4H-pyran-2-yl)thianthren-2-yl)propanamide, or a solvate, salt, N-oxide, or prodrug thereof:

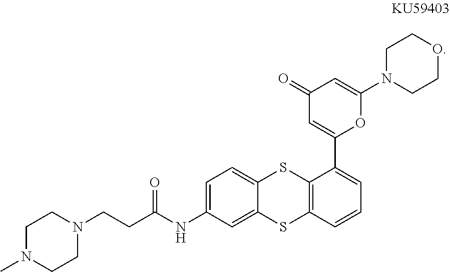 [SMILES: CN1CCN(CCC(=O)Nc2ccc3c(c2)Sc2cccc(-c4cc(=O)cc(N5CCOCC5)o4)c2S3)CC1]

As used herein, the term “KU-60019” or “KU60019” refers to 2-(2,6-dimethylmorpholin-4-yl)-N-(5-(6-morpholin-4-yl-4-oxo-4H-pyran-2-yl)-9H-thioxanthen-2-yl)acetamide, or a solvate, salt, N-oxide, or prodrug thereof:

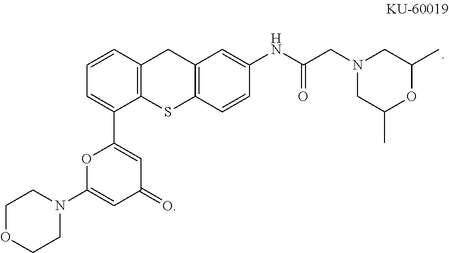 [SMILES: CC1CN(CC(=O)Nc2ccc3c(c2)Cc2cccc(-c4cc(=O)cc(N5CCOCC5)o4)c2S3)CC(C)O1]

As used herein, the term “NSC-109555” or NSC 109555″ refers to 4,4′-diacetyldiphenylurea bis(guanylhydrazone) or a solvate, salt, N-oxide, or prodrug thereof:

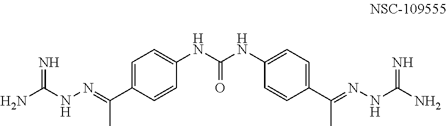 [SMILES: C/C(=N\NC(=N)N)c1ccc(NC(=O)Nc2ccc(/C(C)=N/NC(=N)N)cc2)cc1]

As used herein, a “pharmaceutically acceptable carrier” means a pharmaceutically acceptable material, composition or carrier, such as a liquid or solid filler, diluent, excipient, solvent or encapsulating material, involved in carrying or transporting a compound(s) of the present invention within or to the subject such that it can perform its intended function. Typically, such compounds are carried or transported from one organ, or portion of the body, to another organ, or portion of the body. Each carrier must be “acceptable” in the sense of being compatible with the other ingredients of the formulation, and not injurious to the patient. Some examples of materials that can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. As used herein “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds can also be incorporated into the compositions.

As used herein, the language “pharmaceutically acceptable salt” refers to a salt of the administered compounds prepared from pharmaceutically acceptable non-toxic acids, including inorganic acids, organic acids, solvates, hydrates, or clathrates thereof.

As used herein, the term “PPA” refers to phosphonoacetic acid, or a solvate, salt or prodrug thereof.

As used herein, a viral strain is “resistant” to an antiviral agent if the minimum concentration necessary to inhibit the growth and/or kill the strain is higher than the average minimum concentration that inhibits the growth and/or kills other strains of the same virus. In certain embodiments, the minimum concentration of the antiviral agent necessary to inhibit the growth and/or kill the resistant strain is at least about 2 times higher, about 4 times higher, about 8 times higher, about 16 times higher, about 32 times higher, about 64 times higher, about 128 times higher, about 256 times higher, about 512 times higher, about 1,024 times higher, or about 2,048 times higher, about 10,000 times higher, or about 100,000 times higher than the average minimum concentration of the antiviral agent that inhibits the growth and/or kills other strains of the same virus.

As used herein, the term “SC-203885” refers to (Z)-5-(2-amino-5-oxo-1,5-dihydro-4H-imidazol-4-ylidene)-3,4,5,5a,10,10a-hexahydroazepino[3,4-b]indol-1(2H)-one, or a solvate, salt, N-oxide, or prodrug thereof:

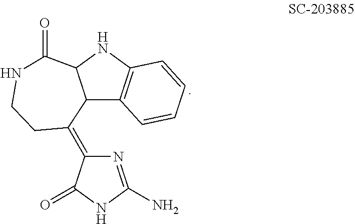 [SMILES: NC1=N/C(=C2/CCNC(=O)C3Nc4ccccc4C23)C(=O)N1]

By the term “specifically bind” or “specifically binds” as used herein is meant that a first molecule (e.g., an antibody) preferentially binds to a second molecule (e.g., a particular antigenic epitope), but does not necessarily bind only to that second molecule.

As used herein, the term “subject” or “patient” or “individual” includes humans and other animals, particularly mammals, and other organisms. Thus the methods are applicable to both human therapy and veterinary applications. In a specific embodiment, the patient is a mammal, and in certain embodiments the patient is human.

As used herein, the term “TK” refers to thymidine kinase.

As used herein, the terms “treat,” “treating,” and “treatment,” refer to therapeutic or preventative measures described herein. The methods of “treatment” employ administration to a subject, in need of such treatment, a composition of the present invention, for example, a subject afflicted a disease or disorder, or a subject who ultimately may acquire such a disease or disorder, in order to prevent, cure, delay, reduce the severity of, or ameliorate one or more symptoms of the disorder or recurring disorder, or in order to prolong the survival of a subject beyond that expected in the absence of such treatment.

Ranges: throughout this disclosure, various aspects of the present invention can be presented in a range format. It should be understood that the description in range format is merely for convenience and brevity and should not be construed as an inflexible limitation on the scope of the present invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention relates generally to compositions and methods for treating diseases and disorders caused by herpes simplex virus type 1, including herpes simplex keratitis, in a subject. In one aspect, the present invention provides a composition for treating herpes simplex keratitis in a subject. In certain embodiments, the compositions of the present invention comprise an ATM inhibitor and an anti-herpetic agent. In other embodiments, the compositions comprise a Chk2 inhibitor and an anti-herpetic agent. In yet other embodiments, the compositions comprise a Chk2 inhibitor and an ATM inhibitor, and optionally an anti-herpetic agent.

In one aspect, the present invention provides a method of treating or preventing herpes simplex keratitis in a subject in need thereof. In certain embodiments, the method comprises administering to the subject an effective amount of a composition comprising an ATM inhibitor and an anti-herpetic agent. In other embodiments, the method comprises administering to the subject an effective amount of a composition comprising a Chk2 inhibitor and an anti-herpetic agent. In yet other embodiments, the method comprises administering to the subject an effective amount of a composition comprising an ATM inhibitor, a Chk2 inhibitor and optionally an anti-herpetic agent. In yet other embodiments, the method comprises administering to the subject an effective amount of an ATM inhibitor and an effective amount of an anti-herpetic agent. In yet other embodiments, the method comprises administering to the subject an effective amount of a Chk2 inhibitor and an effective amount of an anti-herpetic agent. In yet other embodiments, the method comprises administering to the subject an effective amount of a Chk2 inhibitor, an effective amount of an ATM inhibitor and optionally an effective amount of an anti-herpetic agent.

In certain embodiments, administration of an ATM inhibitor reduces the effective amount of the anti-herpetic agent required to be administered to the subject to obtain the same therapeutic benefit. In other embodiments, administration of a Chk2 inhibitor reduces the effective amount of the anti-herpetic agent required to be administered to the subject to obtain the same therapeutic benefit. In yet other embodiments, the reduced effective amount of the anti-herpetic agent required to be administered to the subject to obtain the same therapeutic benefit results in a reduced frequency or severity of side effects due to the anti-herpetic agent experienced by the subject. In yet other embodiments, the infection is caused by a drug-resistant HSV-1 strain. In yet other embodiments, the drug-resistant HSV-1 strain has a TK mutation. In yet other embodiments, the strain is resistant to at least one selected from the group consisting of acyclovir, famciclovir, penciclovir, valacyclovir, acyclovir, trifluridine, penciclovir and valacyclovir.

In certain embodiments, the ATM inhibitor is at least one selected from the group consisting of a nucleic acid, an antisense nucleic acid, an siRNA, a ribozyme, an shRNA, a peptide, an antibody, a small molecule, an antagonist, an aptamer, or a peptidomimetic that reduces the expression or activity of ATM. In other embodiments, the ATM inhibitor is selected from the group consisting of caffeine, wortmannin, chloroquine, CP-466722, KU-55933, KU-59403 and KU-60019, a salt or solvate thereof, and any combinations thereof.

In certain embodiments, the Chk2 inhibitor is at least one selected from the group consisting of a nucleic acid, an antisense nucleic acid, an siRNA, a ribozyme, an shRNA, a peptide, an antibody, a small molecule, an antagonist, an aptamer, or a peptidomimetic that reduces the expression or activity of Chk2. In other embodiments, the Chk2 inhibitor is Chk2 inhibitor II, SC-203885 or NSC-109555.

In certain embodiments, the anti-herpetic agent is at least one selected from the group consisting of acyclovir, famciclovir, penciclovir, valacyclovir, acyclovir, trifluridine, penciclovir and valacyclovir.

In certain embodiments, the composition comprises a combination of inhibitors described herein. For example, in certain embodiments the composition comprises a combination of an ATM inhibitor and a Chk2 inhibitor, in combination with an optional anti-herpetic agent.

In one aspect, the present studies shed light on the concept of interfering with the host DDR in order to suppress corneal herpesvirus infection. The traditional approach of inhibiting critical viral proteins, such as DNA polymerase, has clear limitations. Analogous to antibiotic drugs, antiviral compounds that specifically target a viral factor leave room for mutation-driven development of resistance. This is a well-recognized emerging clinical problem, particularly in immunosuppressed populations. The most common mechanism of resistance to nucleoside analogues (˜95%) is mutation of the viral TK gene. By contrast, disruption of a critical virus-host interaction via inhibition of a host factor suppresses viral replication without the risk of rapid development of mutation-based resistance.

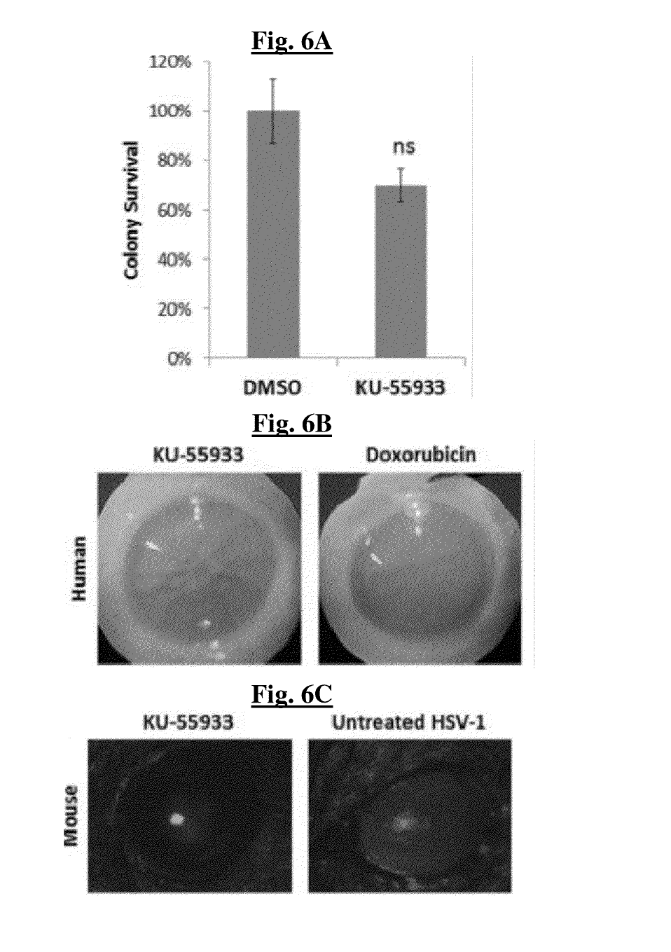

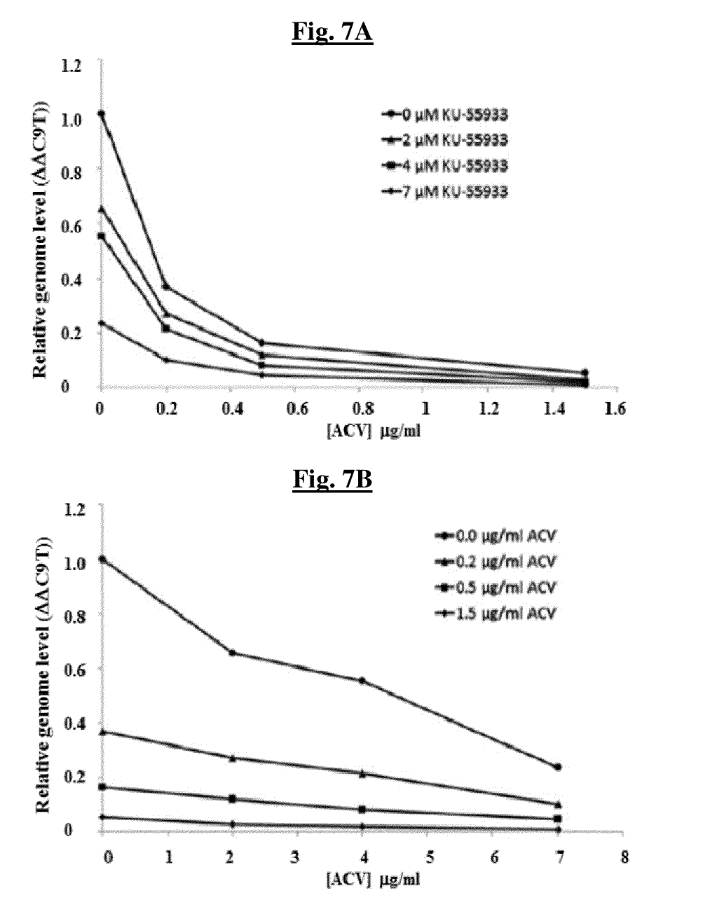

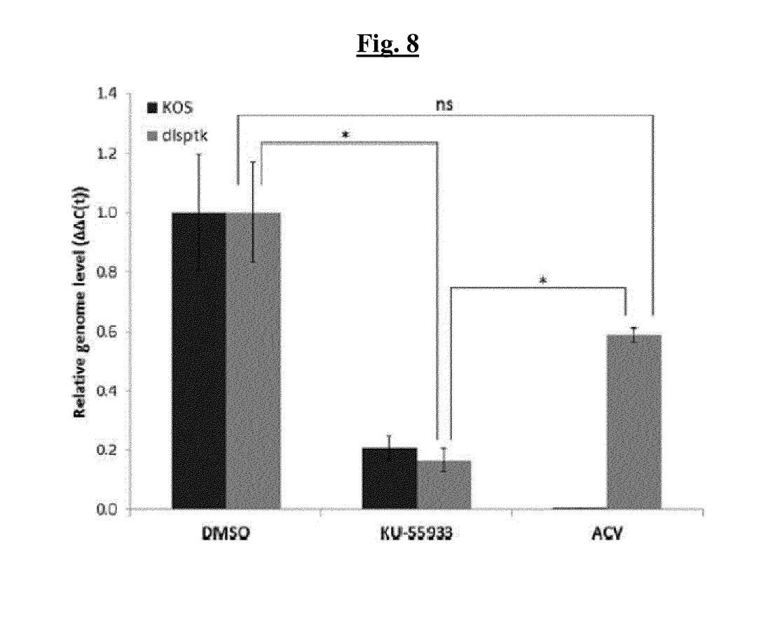

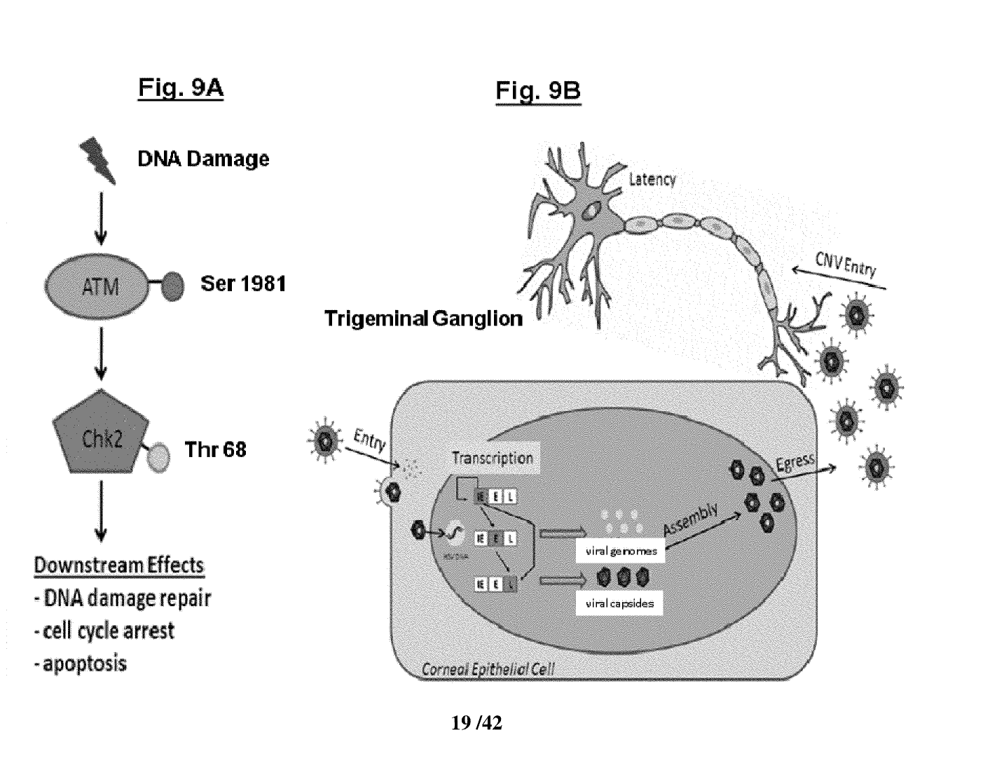

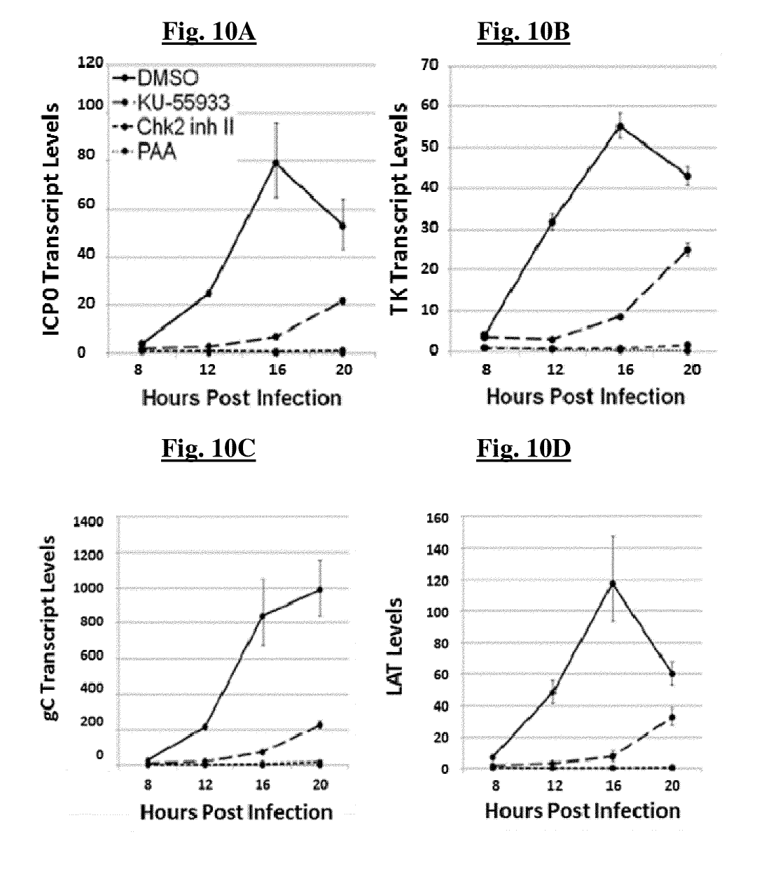

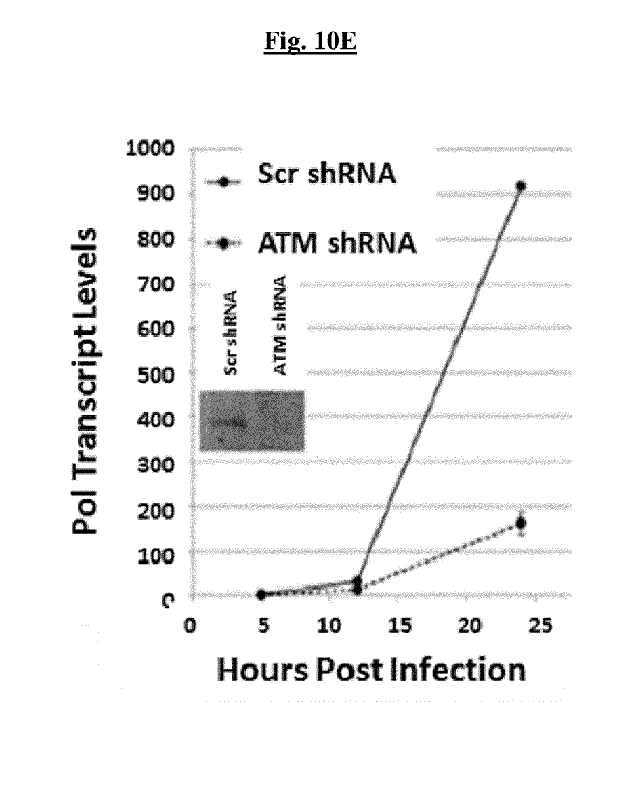

In certain embodiments of the present invention, ATM inhibitors are combined with established antiviral agents in the treatment of herpes keratitis. Without wishing to be limited by any theory, the diversification of targeted pathways accomplished by combination therapy has the two-fold advantage of preventing resistance and allowing for a reduction in drug dosage, with a consequent attenuation of side effect severity of each individual drug. The present experiments with resistant infection (FIG. 8) and combination treatments (FIGS. 7A-7B) demonstrate that inhibition of ATM offer these advantages in the treatment of herpes keratitis.

The examination of the corneal toxicity of KU-55933 revealed a generally favorable toxicity profile in cultured cells, which were able to survive and proliferate well for 2 full weeks following a 24-hour treatment (FIG. 6A). In line with this result, explanted human corneas did not develop any surface defects following a continuous 24-hour treatment with KU-55933 (FIG. 6B). Mice that had received prolonged topical KU-55933 treatment for 4 full days (every 4 hours for the first day and every 8 hours for the next 3 days) did not show epithelial abnormalities by fluorescein staining. The present results indicate that ATM inhibitors are sufficiently safe for topical application to the cornea.

In summary, the present work highlights the DDR as a promising area for potential antiviral targets in the treatment of herpes keratitis. In certain embodiments, ATM inhibitors may be used in combination therapy to reduce the toxicity of topical antivirals, and as standalone therapy against drug-resistant HSV-1 strains.

In another aspect, the present invention sheds light on the mechanism whereby ATM activation facilitates HSV-1 replication in the cornea. Chk2 kinase is a widely-recognized signaling target of ATM, and the present disclosure highlights the significance of Chk2 activity in corneal epithelial HSV-1 infection. As demonstrated herein, blocking Chk2 kinase activity with a small molecule inhibitor produced pronounced inhibition of infection in two different human corneal epithelial cell lines. This inhibition was detectible by monitoring viral genome levels, production of infectious viral particles, and visually by observing the cytopathic effect of the virus. In addition, these in vitro findings were extended into the ex vivo model of corneal epithelial keratitis, where Chk2 inhibition blocked viral replication in human and rabbit corneas. These findings expand the knowledge on the role of the DDR in the pathogenesis of HK, and establish Chk2 kinase as a significant factor that mediates the pro-viral effect of ATM activation in corneal epithelial HSV-1 infection.

The present disclosure establishes Chk2 kinase activity as a critical factor in the interaction between HSV-1 and the host DDR, and sheds light on the role of ATM signaling in the molecular pathology of HK. In certain embodiments, the corneal toxicity profile of Chk2 inhibitors allows for their use in therapeutic treatment.

### Inhibitors

In certain embodiments, the compositions of the present invention comprise an ATM inhibitor. An ATM inhibitor is any compound or molecule that reduces, inhibits, or prevents the function of ATM. For example, an ATM inhibitor is any compound or molecule that reduces ATM expression, activity, or both. In certain embodiments, an ATM inhibitor comprises at least one selected from the group consisting of a nucleic acid, an antisense nucleic acid, an siRNA, a ribozyme, an shRNA, a peptide, an antibody, a small molecule, an antagonist, an aptamer, and a peptidomimetic.

In certain embodiments, the composition of the present invention comprises an Chk2 inhibitor. A Chk2 inhibitor is any compound or molecule that reduces, inhibits, or prevents the function of Chk2. For example, a Chk2 inhibitor is any compound or molecule that reduces Chk2 expression, activity, or both. In certain embodiments, a Chk2 inhibitor comprises at least one selected from the group consisting of a nucleic acid, an antisense nucleic acid, an siRNA, a ribozyme, an shRNA, a peptide, an antibody, a small molecule, an antagonist, an aptamer, and a peptidomimetic.

In certain embodiments, the compositions of the present invention comprises a pharmaceutically acceptable carrier.

### Small Molecule Inhibitors

In certain embodiments, the inhibitor is a small molecule. When the inhibitor is a small molecule, a small molecule may be obtained using standard methods known to the skilled artisan. Such methods include chemical organic synthesis or biological means. Biological means include purification from a biological source, recombinant synthesis and in vitro translation systems, using methods well known in the art. In certain embodiments, a small molecule inhibitor of the present invention comprises an organic molecule, an inorganic molecule, a biomolecule, and the like.

Combinatorial libraries of molecularly diverse chemical compounds potentially useful in treating a variety of diseases and conditions are well known in the art as are method of making the libraries. The method may use techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores.

Small molecule inhibitors of ATM are known in the art. Exemplary small molecule ATM inhibitors include, but are not limited to caffeine, wortmannin, chloroquine, CP-466722, KU-55933, KU-59403 or KU-60019. Exemplary small molecule Chk2 inhibitors include, but are not limited to Chk2 inhibitor II, SC-203885 or NSC-109555.

Where tautomeric forms may be present for any of the inhibitors described herein, each and every tautomeric form is intended to be included in the present invention, even though only one or some of the tautomeric forms may be explicitly illustrated.

The invention also includes any or all of the stereochemical forms, including any enantiomeric or diasteriomeric forms of the inhibitors described. The recitation of the structure or name herein is intended to embrace all possible stereoisomers of inhibitors depicted. All forms of the inhibitors are also embraced by the invention, such as crystalline or non-crystalline forms of the inhibitors. Compositions comprising an inhibitor of the present invention are also intended, such as a composition of substantially pure inhibitor, including a specific stereochemical form thereof, or a composition comprising mixtures of inhibitors of the present invention in any ratio, including two or more stereochemical forms, such as in a racemic or non-racemic mixture. In certain embodiments, the small molecule inhibitor of the present invention comprises an analog or derivative of an inhibitor described herein.

In certain embodiments, the small molecules described herein are candidates for derivatization. In certain embodiments, the analogs of the small molecules described herein that have modulated potency, selectivity, and solubility are included herein and provide useful leads for drug discovery and drug development. Thus, in certain instances, during optimization new analogs are designed considering issues of drug delivery, metabolism, novelty, and safety.

In some instances, small molecule inhibitors described herein are derivatized/analoged as is well known in the art of combinatorial and medicinal chemistry. The analogs or derivatives can be prepared by adding and/or substituting functional groups at various locations. As such, the small molecules described herein can be converted into derivatives/analogs using well known chemical synthesis procedures. For example, all of the hydrogen atoms or substituents can be selectively modified to generate new analogs. Also, the linking atoms or groups can be modified into longer or shorter linkers with carbon backbones or hetero atoms. Also, the ring groups can be changed so as to have a different number of atoms in the ring and/or to include hetero atoms. Moreover, aromatics can be converted to cyclic rings, and vice versa. For example, the rings may be from 5-7 atoms, and may be homocycles or heterocycles.

In certain embodiments, the small molecule inhibitors described herein can independently be derivatized/analoged by modifying hydrogen groups independently from each other into other substituents. That is, each atom on each molecule can be independently modified with respect to the other atoms on the same molecule. Any traditional modification for producing a derivative/analog can be used. For example, the atoms and substituents can be independently comprised of hydrogen, an alkyl, aliphatic, straight chain aliphatic, aliphatic having a chain hetero atom, branched aliphatic, substituted aliphatic, cyclic aliphatic, heterocyclic aliphatic having one or more hetero atoms, aromatic, heteroaromatic, polyaromatic, polyamino acids, peptides, polypeptides, combinations thereof, halogens, halo-substituted aliphatics, and the like. Additionally, any ring group on a compound can be derivatized to increase and/or decrease ring size as well as change the backbone atoms to carbon atoms or hetero atoms.

### Nucleic Acid Inhibitors

In certain embodiments, the invention includes an isolated nucleic acid. In other embodiments, the inhibitor is an siRNA, shRNA or antisense molecule, which inhibits ATM or Chk2. In certain embodiments, the nucleic acid comprises a promoter/regulatory sequence such that the nucleic acid is preferably capable of directing expression of the nucleic acid. Thus, the invention encompasses expression vectors and methods for the introduction of exogenous DNA into cells with concomitant expression of the exogenous DNA in the cells such as those described, for example, in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York) and as described elsewhere herein.

In certain embodiments, ATM or Chk2 can be inhibited by way of inactivating and/or sequestering ATM or Chk2. As such, inhibiting the activity of ATM or Chk2 can be accomplished by using a transdominant negative mutant.

In certain embodiments, a nucleic acid is used to decrease the level of ATM or Chk2 protein. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(19):306-311; Timmons et al., 1998, Nature 395:854; Montgomery et al., 1998, TIG 14 (7):255-258; David R. Engelke, Ed., RNA Interference (RNAi) Nuts & Bolts of RNAi Technology, DNA Press, Eagleville, Pa. (2003); and Gregory J. Hannon, Ed., RNAi A Guide to Gene Silencing, Cold Spring Harbor Laboratory Press, Cold Spring Harbor, N.Y. (2003). Soutschek et al. (2004, Nature 432:173-178) describe a chemical modification to siRNAs that aids in intravenous systemic delivery. Optimizing siRNAs involves consideration of overall G/C content, C/T content at the termini, Tₘ and the nucleotide content of the 3′ overhang. See, for instance, Schwartz et al., 2003, Cell, 115:199-208 and Khvorova et al., 2003, Cell 115:209-216. Therefore, the present invention also includes methods of decreasing levels of ATM or Chk2 using RNAi technology.

In another aspect, the invention includes a vector comprising an siRNA or antisense nucleic acid. Preferably, the antisense nucleic acid is capable of inhibiting the expression of a target polypeptide, wherein the target polypeptide is selected from the group consisting of ATM and Chk2. The incorporation of a desired polynucleotide into a vector and the choice of vectors is well-known in the art as described in, for example, Sambrook et al. (2012), and in Ausubel et al. (1997), and elsewhere herein.

In certain embodiments, the expression vectors described herein encode a short hairpin RNA (shRNA) inhibitor. shRNA inhibitors are well known in the art and are directed against the mRNA of a target, thereby decreasing the expression of the target. In certain embodiments, the encoded shRNA is expressed by a cell, and is then processed into siRNA. For example, in certain instances, the cell possesses native enzymes (e.g., dicer) that cleaves the shRNA to form siRNA.

The siRNA, shRNA, or antisense nucleic acid can be cloned into a number of types of vectors as described elsewhere herein. For expression of the siRNA or antisense polynucleotide, at least one module in each promoter functions to position the start site for RNA synthesis.

In order to assess the expression of the siRNA, shRNA, or antisense polynucleotide, the expression vector to be introduced into a cell can also contain either a selectable marker gene or a reporter gene or both to facilitate identification and selection of expressing cells from the population of cells sought to be transfected or infected using a viral vector. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neomycin resistance and the like.

Therefore, in another aspect, the invention relates to a vector, comprising the nucleotide sequence of the present invention or the construct of the present invention. The choice of the vector will depend on the host cell in which it is to be subsequently introduced. In certain embodiments, the vector of the present invention is an expression vector. Suitable host cells include a wide variety of prokaryotic and eukaryotic host cells. In other embodiments, the expression vector is selected from the group consisting of a viral vector, a bacterial vector and a mammalian cell vector. Prokaryote- and/or eukaryote-vector based systems can be employed for use with the present invention to produce polynucleotides, or their cognate polypeptides. Many such systems are commercially and widely available.

Further, the expression vector may be provided to a cell in the form of a viral vector. Viral vector technology is well known in the art and is described, for example, in Sambrook et al. (2012), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

By way of illustration, the vector in which the nucleic acid sequence is introduced can be a plasmid that is or is not integrated in the genome of a host cell when it is introduced in the cell. Illustrative, non-limiting examples of vectors in which the nucleotide sequence of the present invention or the gene construct of the present invention can be inserted include a tet-on inducible vector for expression in eukaryote cells. The vector may be obtained by conventional methods known by persons skilled in the art (Sambrook et al., 2012). In a particular embodiment, the vector is a vector useful for transforming animal cells.

In certain embodiments, the recombinant expression vectors may also contain nucleic acid molecules which encode a peptide or peptidomimetic inhibitor of invention, described elsewhere herein.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. No. 4,683,202, U.S. Pat. No. 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2012). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

The recombinant expression vectors may also contain a selectable marker gene which facilitates the selection of transformed or transfected host cells. Suitable selectable marker genes are genes encoding proteins such as G418 and hygromycin that confer resistance to certain drugs, β-galactosidase, chloramphenicol acetyltransferase, firefly luciferase, or an immunoglobulin or portion thereof such as the Fc portion of an immunoglobulin preferably IgG. The selectable markers may be introduced on a separate vector from the nucleic acid of interest.

Following the generation of the antisense nucleic acid, a skilled artisan will understand that the antisense nucleic acid will have certain characteristics that can be modified to improve the antisense nucleic acid as a therapeutic compound. Therefore, the antisense nucleic acid may be further designed to resist degradation by modifying it to include phosphorothioate, or other linkages, methylphosphonate, sulfone, sulfate, ketyl, phosphorodithioate, phosphoramidate, phosphate esters, and the like (see, e.g., Agrwal et al., 1987, Tetrahedron Lett. 28:3539-3542; Stec et al., 1985 Tetrahedron Lett. 26:2191-2194; Moody et al., 1989 Nucleic Acids Res. 12:4769-4782; Eckstein, 1989 Trends Biol. Sci. 14:97-100; Stein, In: Oligodeoxynucleotides. Antisense Inhibitors of Gene Expression, Cohen, ed., Macmillan Press, London, pp. 97-117 (1989)).

Any polynucleotide may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queosine, and wybutosine and the like, as well as acetyl-methyl-, thio- and other modified forms of adenine, cytidine, guanine, thymine, and uridine.

In certain embodiments of the present invention, an antisense nucleic acid sequence that is expressed by a plasmid vector is used to inhibit ATM or Chk2 protein expression. The antisense expressing vector is used to transfect a mammalian cell or the mammal itself, thereby causing reduced endogenous expression of ATM or Chk2.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the present invention may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243).

In certain embodiments of the present invention, a ribozyme is used to inhibit ATM or Chk2 protein expression. Ribozymes useful for inhibiting the expression of a target molecule may be designed by incorporating target sequences into the basic ribozyme structure which are complementary, for example, to the mRNA sequence encoding ATM or Chk2. Ribozymes targeting ATM or Chk2, may be synthesized using commercially available reagents (Applied Biosystems, Inc., Foster City, Calif.) or they may be genetically expressed from DNA encoding them.

### Polypeptide Inhibitors

In certain embodiments, the invention includes an isolated peptide inhibitor that inhibits ATM or Chk2. In other embodiments, the peptide inhibitor of the present invention inhibits ATM or Chk2 directly by binding to ATM or Chk2, thereby preventing the normal functional activity of ATM or Chk2. In yet other embodiments, the peptide inhibitor of the present invention inhibits ATM or Chk2 by competing with endogenous ATM or Chk2. In yet other embodiments, the peptide inhibitor of the present invention inhibits the activity of ATM or Chk2 by acting as a transdominant negative mutant.

The variants of the polypeptides according to the present invention may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present invention, (iv) fragments of the polypeptides, and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

### Antibody Inhibitors

The invention also contemplates an inhibitor of ATM or Chk2 comprising an antibody, or antibody fragment, specific for ATM or Chk2. That is, the antibody can inhibit ATM or Chk2 to provide a beneficial effect.

The antibodies may be intact monoclonal or polyclonal antibodies, and immunologically active fragments (e.g., a Fab or (Fab)₂ fragment), an antibody heavy chain, an antibody light chain, humanized antibodies, a genetically engineered single chain Fᵥ molecule (Ladner et al, U.S. Pat. No. 4,946,778), or a chimeric antibody, for example, an antibody that contains the binding specificity of a murine antibody, but in which the remaining portions are of human origin. Antibodies including monoclonal and polyclonal antibodies, fragments and chimeras, may be prepared using methods known to those skilled in the art.

Antibodies can be prepared using intact polypeptides or fragments containing an immunizing antigen of interest. The polypeptide or oligopeptide used to immunize an animal may be obtained from the translation of RNA or synthesized chemically and can be conjugated to a carrier protein, if desired. Suitable carriers that may be chemically coupled to peptides include bovine serum albumin and thyroglobulin, keyhole limpet hemocyanin. The coupled polypeptide may then be used to immunize the animal (e.g., a mouse, a rat, or a rabbit).

### Methods

In one aspect, the present invention provides a method of treating or preventing herpes simplex keratitis in a subject in need thereof. In certain embodiments, the method comprises administering to the subject an effective amount of a composition comprising an ATM inhibitor and an anti-herpetic agent. In other embodiments, the method comprises administering to the subject an effective amount of a composition comprising a Chk2 inhibitor and an anti-herpetic agent. In yet other embodiments, the method comprises administering to the subject an effective amount of a composition comprising an ATM inhibitor, a Chk2 inhibitor and optionally an anti-herpetic agent. In yet other embodiments, the method comprises administering to the subject an effective amount of an ATM inhibitor and an effective amount of an anti-herpetic agent. In yet other embodiments, the method comprises administering to the subject an effective amount of a Chk2 inhibitor and an effective amount of an anti-herpetic agent. In yet other embodiments, the method comprises administering to the subject an effective amount of a Chk2 inhibitor, an effective amount of an ATM inhibitor and optionally an effective amount of an anti-herpetic agent. In yet other embodiments, the compositions of the present invention comprise a pharmaceutically acceptable carrier.

In certain embodiments, administration of an ATM inhibitor reduces the effective amount of the anti-herpetic agent required to be administered to the subject to obtain the same therapeutic benefit. In other embodiments, administration of a Chk2 inhibitor reduces the effective amount of the anti-herpetic agent required to be administered to the subject to obtain the same therapeutic benefit. In yet other embodiments, the reduced effective amount of the anti-herpetic agent required to be administered to the subject to obtain the same therapeutic benefit results in a reduced frequency or severity of side effects due to the anti-herpetic agent experienced by the subject. In yet other embodiments, the infection is caused by a drug-resistant HSV-1 strain. In yet other embodiments, the drug-resistant HSV-1 strain has a TK mutation. In yet other embodiments, the strain is resistant to at least one selected from the group consisting of acyclovir, famciclovir, penciclovir, valacyclovir, acyclovir, trifluridine, penciclovir and valacyclovir.

In certain embodiments, the ATM inhibitor is at least one selected from the group consisting of a nucleic acid, an antisense nucleic acid, an siRNA, a ribozyme, an shRNA, a peptide, an antibody, a small molecule, an antagonist, an aptamer, or a peptidomimetic that reduces the expression or activity of ATM. In other embodiments, the ATM inhibitor is selected from the group consisting of caffeine, wortmannin, chloroquine, CP-466722, KU-55933, KU-59403 and KU-60019, a salt or solvate thereof, and any combinations thereof.

In certain embodiments, the Chk2 inhibitor is at least one selected from the group consisting of a nucleic acid, an antisense nucleic acid, an siRNA, a ribozyme, an shRNA, a peptide, an antibody, a small molecule, an antagonist, an aptamer, or a peptidomimetic that reduces the expression or activity of Chk2. In other embodiments, the Chk2 inhibitor is Chk2 inhibitor II, SC-203885 or NSC-109555.

In certain embodiments, the anti-herpetic agent is at least one selected from the group consisting of acyclovir, famciclovir, penciclovir, valacyclovir, acyclovir, trifluridine, penciclovir and valacyclovir.

In certain embodiments, the composition comprises a combination of inhibitors described herein. For example, in certain embodiments the composition comprises a combination of an ATM inhibitor and a Chk2 inhibitor, in combination with an optional anti-herpetic agent.

ATM or Chk2 activity can be inhibited using any method known to the skilled artisan. Examples of methods that inhibit ATM or Chk2 activity, include but are not limited to, inhibiting expression of an endogenous gene encoding ATM or Chk2, decreasing expression of mRNA encoding ATM or Chk2, and inhibiting the function, activity, or stability of ATM or Chk2. An ATM or Chk2 inhibitor may therefore be a compound that decreases expression of a gene encoding ATM or Chk2, decreases RNA half-life, stability, or expression of a mRNA encoding ATM or Chk2 protein, or inhibits ATM or Chk2 function, activity or stability. An ATM or Chk2 inhibitor may be any type of compound, including but not limited to, a peptide, a nucleic acid, an antisense nucleic acid, an aptamer, a peptidometic, and a small molecule, or combinations thereof.

ATM or Chk2 inhibition may be accomplished either directly or indirectly. For example ATM or Chk2 may be directly inhibited by compounds or compositions that directly interact with ATM or Chk2, such as antibodies. Alternatively, ATM or Chk2 may be inhibited indirectly by compounds or compositions that inhibit ATM or Chk2 downstream effectors, or upstream regulators which up-regulate ATM or Chk2 expression.

Decreasing expression of an endogenous gene includes providing a specific inhibitor of gene expression. Decreasing expression of mRNA or protein includes decreasing the half-life or stability of mRNA or decreasing expression of mRNA. Methods of decreasing expression of ATM or Chk2 include, but are not limited to, methods that use an siRNA, a microRNA, an antisense nucleic acid, a ribozyme, an expression vector encoding a transdominant negative mutant, a peptide, a small molecule, and combinations thereof.

### Administration

The invention also encompasses the use of pharmaceutical compositions of at least one composition of the present invention or a salt thereof to practice the methods of the present invention. Such a pharmaceutical composition may consist of at least one composition of the present invention or a salt thereof, in a form suitable for administration to a subject, or the pharmaceutical composition may comprise at least one composition of the present invention or a salt thereof, and one or more pharmaceutically acceptable carriers, one or more additional ingredients, or some combination of these. The at least one composition of the present invention may be present in the pharmaceutical composition in the form of a physiologically acceptable salt, such as in combination with a physiologically acceptable cation or anion, as is well known in the art.

Administration of an ATM inhibitor, a Chk2 inhibitor, or an anti-herpetic agent in a method of treatment can be achieved in a number of different ways, using methods known in the art. The relative amounts of the active ingredient, the pharmaceutically acceptable carrier, and any additional ingredients in a pharmaceutical composition of the present invention will vary, depending upon the identity, size, and condition of the subject treated and further depending upon the route by which the composition is to be administered.

In certain embodiments, the composition is administered to the subject by an intrapulmonary, intrabronchial, inhalational, intranasal, intratracheal, intravenous, intramuscular, subcutaneous, topical, transdermal, oral, buccal, rectal, pleural, peritoneal, vaginal, epidural, otic, intraocular, or intrathecal route. In other embodiments, the composition is administered to the subject by a topical or intraocular route.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

In various embodiments, an ATM inhibitor and an anti-herpetic agent, or a Chk2 inhibitor and an anti-herpetic agent, are administered to a subject. The inhibitor may also be a hybrid or fusion composition to facilitate, for instance, delivery to target cells or efficacy. In certain embodiments, a hybrid composition may comprise a tissue-specific targeting sequence.

The therapeutic and prophylactic methods of the present invention thus encompass the use of pharmaceutical compositions of the present invention to practice the methods of the present invention. The pharmaceutical compositions useful for practicing the invention may be administered to deliver a dose to the subject of from 1 ng/kg/day and 100 mg/kg/day. In certain embodiments, the invention envisions administration of a dose which results in a concentration of the compound of the present invention from 1 μM and 10 μM in a mammal.

Typically, dosages which may be administered in a method of the present invention to a mammal, preferably a human, range in amount from 0.5 μg to about 50 mg per kilogram of body weight of the mammal, while the precise dosage administered will vary depending upon any number of factors, including but not limited to, the type of mammal and type of disease state being treated, the age of the mammal and the route of administration. Preferably, the dosage of the compound will vary from about 1 μg to about 10 mg per kilogram of body weight of the mammal. More preferably, the dosage will vary from about 3 μg to about 1 mg per kilogram of body weight of the mammal.

Compositions of the present invention for administration may be in the range of from about 1 μg to about 1,000 mg, about 2 μg to about 500 mg, about 4 μg to about 250 mg, about 6 μg to about 200 mg, about 8 μg to about 100 mg, about 10 μg to about 50 mg, about 20 μg to about 25 mg, about 40 μg to about 10 mg, about 50 μg to about 5 mg, about 100 μg to about 1 mg, and any and all whole or partial increments thereinbetween.

In some embodiments, the dose of a composition of the present invention is from about 0.5 μg and about 2,000 mg. In some embodiments, a dose of a composition described herein is less than about 2,000 mg, or less than about 1,000 mg, or less than about 500 mg, or less than about 250 mg, or less than about 100 mg, or less than about 50 mg, or less than about 25 mg, or less than about 10 mg, or less than about 5 mg, or less than about 1 mg, and any and all whole or partial increments thereof.

The compound may be administered to a mammal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the mammal, etc.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition.

Suitable compositions and dosage forms include, for example, suspensions, granules, beads, powders, pellets, and liquid sprays for nasal administration, dry powder or aerosolized formulations for inhalation, and the like. It should be understood that the formulations and compositions that would be useful in the present invention are not limited to the particular formulations and compositions that are described herein. For example, formulations may comprise a powder or an aerosolized or atomized solution or suspension comprising the active ingredient. Such powdered, aerosolized, or aerosolized formulations may further comprise one or more of the additional ingredients described herein. The examples of formulations described herein are not exhaustive and it is understood that the invention includes additional modifications of these and other formulations not described herein, but which are known to those of skill in the art.

In certain embodiments, the invention includes a method comprising administering a combination of a kinase inhibitor and an anti-herpetic agent elsewhere described herein. In certain embodiments, the method has an additive effect, wherein the overall effect of the administering a combination of a kinase inhibitor and an anti-herpetic agent is approximately equal to the sum of the effects of administering each of the inhibitor or anti-herpetic agent alone. In other embodiments, the method has a synergistic effect, wherein the overall effect of administering a combination of a kinase inhibitor and an anti-herpetic agent is greater than the sum of the effects of administering each of the inhibitor or anti-herpetic agent alone.

The method comprises administering a combination of a kinase inhibitor and an anti-herpetic agent in any suitable ratio. For example, in various embodiments, the method comprises administering the inhibitor and the anti-herpetic agent at a 500:1 ratio, a 100:1 ratio, a 50:1 ration, a 10:1 ratio, a 1:1 ratio, a 1:10 ratio, a 1:50 ratio, a 1:100 ratio, or a 1:500, or any ratio therebetween. However, the method is not limited to any particular ratio. Rather, any ratio that is shown to be effective is encompassed.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

### Materials and Methods

### Cells and Viruses

All cells were cultured at 37° C. and 5% CO₂ and supplemented with 100 U/mL penicillin and 100 U/mL streptomycin. Human corneal epithelial cells immortalized with hTERT (hTCEpi; Robertson, et al., 2005, Invest Ophthalmol V is Sci. 46:470-478) were grown in complete keratinocyte growth medium 2 (KGM-2; Lonza, Basel, Switzerland). African green monkey kidney fibroblasts (CV-1; American Type Culture Collection, Manassas, Va.) were grown in Dulbecco's modified Eagle's medium (DMEM; Cellgro, Manassas, Va.) supplemented with 10% FBS. The KOS strain of HSV-1 was used in the in vitro and ex vivo infections, whereas McKrae strain was used for in vivo mouse experiments, and TK mutant dlsptk strain (Coen et al., 1989, Proc Natl Acad Sci USA 86:4736-4740) was used in the drug resistance experiments. All viral stocks were titered on CV-1 monolayers.

### Infection and Treatments of Cultured Cells

The following strains of HSV-1 were used: KOS, ICP0-null, HFEM, and tsB7.

Subconfluent monolayers of cells were grown in six-well plates. Drug treatments were administered 45 minutes prior to infection and continued for the entire duration of each experiment. Unless indicated otherwise, KU-55933 (Batch No. 5, 99.7% purity; Tocris Bioscience, Bristol, UK) was used at 10 μM final concentration, phosphonoacetic acid (PAA) at 400 μg/mL (Sigma-Aldrich, St. Louis, Mo.), and acyclovir at 50 μg/mL (Sigma-Aldrich). KU-55933 was dissolved in dimethyl sulfoxide (DMSO), and the final concentration of DMSO in both KU-55933 and mock treatment in the in vitro and the ex vivo experiments was 0.1%.

Infections with KOS strain of HSV-1 were carried out in six-well plates in a 200 μL inoculum volume at 37° C. for 1 hour with intermittent rocking. The cells were then rinsed and overlaid with fresh medium.

### Corneal Explant Model

Human corneas were obtained from the Lions Eye Bank of Delaware Valley. Rabbit corneas were excised from intact fresh eyeballs of young (8-12 weeks) albino rabbits (Pel-Freez Biologicals, Rogers, Ark.). Protocol (Alekseev et al., 2012, J Vis Exp 69:e3631) for ex vivo corneal culture, infection, and treatment was followed closely. Briefly, corneoscleral buttons were excised and rinsed in PBS containing 200 U/mL penicillin and 200 μg/mL streptomycin. The endothelial concavity was filled with culture medium containing 1% low melting temperature agarose. The corneas were cultured epithelial side up in MEM medium supplemented with nonessential amino acids (1×), 2 mM L-glutamate, 200 U/mL penicillin, and 200 μg/mL streptomycin. The next day, they were infected with 1×10⁴ plaque forming units (PFU)/cornea of strain KOS HSV-1 for 1 hour, rinsed, and overlaid with fresh medium. Drug treatments were administered at the same concentrations as for cultured cells. For KU-55933 bioavailability assessment, corneas were treated with bleomycin (200 μg/mL) for 1 hour. The epithelial cell layer was collected by scraping to isolate DNA or protein. For immunohistochemistry studies, corneas were flash frozen in optimal cutting temperature (OCT) compound, sectioned, and immunostained using standard protocols. Treatment toxicity was assessed by briefly staining the cornea with fluorescein (1% wt/vol in PBS) and imaging the epithelial defects with 464-nm wavelength blue light (LDP LLC, Carlstadt, N.J.).

### Mouse Ocular Infection and Treatments

Four-week-old female C57BL/6J mice were anesthetized with isoflurane, and their left eyes were scarified in a 4×4 crosshatch pattern with a 28-gauge needle. McKrae strain HSV-1 was applied in 1 μL inoculum volume at 8×10⁵ PFU/eye and the eyelid gently massaged. The infection was allowed to develop for 24 hours, at which point treatments were initiated. KU-55933 was delivered to the corneas dissolved in PBS to a concentration of 200 μM. Control treatments constituted an equivalent amount of DMSO (0.2% vol/vol) in PBS drops. Treatments were administered every 4 hours for 1 full day and then every 8 hours for the remainder of the experiment.

### Disease Scoring

Ocular disease severity was assessed at every 24-hour period postinfection. Two disease parameters were scored based on a number scale (Jose et al., 2013, Invest Ophthalmol V is Sci 54:1070-1079). Briefly, stromal keratitis was scored as 1+, cloudiness, some iris detail visible; 2+, iris detail obscured; 3+, cornea totally opaque; and 4+, corneal perforation. Blepharitis was scored as 1+, puffy eyelids; 2+, puffy eyelids with some crusting; 3+, eye swollen shut with severe crusting; and 4+, eye completely swollen shut and crusted over.

### Viral Genome Replication and Transcription

Viral genome replication and transcription were measured by quantitative PCR (qPCR). Total DNA and RNA from infected cells were isolated using the DNeasy Blood & Tissue Kit and the RNeasy Mini Kit, respectively (QIAGEN, Hilden, Germany). RNA was converted to cDNA using qScript (Quanta BioSciences, Gaithersburg, Md.). Real-time qPCR was performed with SYBR Green (Bio-Rad, Hercules, Calif.). Target primers for UL30 (DNA polymerase catalytic subunit) and reference primers for glyceraldehyde 3-phosphate dehydrogenase (GAPDH) were used to measure genome replication. Transcription of the three gene families was measured with primers for RL2 (ICP0), UL30 (DNA polymerase catalytic subunit), and UL44 (gC), with reference primers for the 18S ribosomal RNA (rRNA). All primer sequences are listed in Table 1.

### Immunocytochemistry and Immunohistochemistry

For immunocytochemistry analysis, cells were grown on cover slips and infected as indicated. Cells were fixed in 3% paraformaldehyde/2% sucrose solution for 10 minutes and permeabilized with 0.5% Triton X-100 for 5 minutes. For immunohistochemistry, corneas were flash frozen in OCT compound and sectioned at 10-μm thickness. Indirect immunofluorescence was performed with primary antibodies against ICP8 (rabbit polyclonal), pATM S1981 (mouse monoclonal; Rockland, Glibertsville, Pa.), and cleaved caspase 3 (rabbit polyclonal; Cell Signaling, Danvers, Mass.). Nuclei were counterstained with Hoechst 33258 (Sigma-Aldrich).

### Western Blot

Standard protocol was followed for Western blot analysis. Cell lysates were collected in 200 μL Laemmli buffer, vortexed, and boiled at 95° C. for 5 minutes. Protein concentrations were measured by bicinchoninic acid (BCA) assay. SDS-PAGE was followed by transfer onto a polyvinylidene difluoride (PVDF) membrane, which was then blocked in 5% BSA. Primary antibodies against the following proteins were used: ICP0 (mouse monoclonal; Virusys Corporation, Taneytown, Md.), ICP4 and nucleolin (both mouse monoclonal; Santa Cruz Biotechnology, Santa Cruz, Calif.), ICP8 (rabbit polyclonal), glycoprotein B and C (mouse monoclonal and rabbit polyclonal, respectively), ATM and pATM S1981 (rabbit polyclonal and mouse monoclonal, respectively; Rockland), Chk2 and pChk2 T68 (rabbit polyclonal and mouse monoclonal, respectively; Cell Signaling). Blots were stained with secondary antibodies and visualized with the Odyssey near-infrared system (LI-COR, Lincoln, Nebr.).

### Colony Survival Assay

hTCEpi cells were treated with KU-55933 (10 μM) or DMSO for 24 hours, trypsinized, counted, and plated into 6-cm dishes at 50 cells/dish. After 2 weeks, colonies were fixed with 10% buffered formalin, stained with 0.01% crystal violet, rinsed, and counted.

### Statistical Analysis

Statistical significance was determined using Student's t-test and is indicated as ns (P>0.05), *(P<0.05), **(P<0.01), or ***(P<0.001).

### Example 1

### HSV-1 Induces ATM Activation in Corneal Epithelial Cells

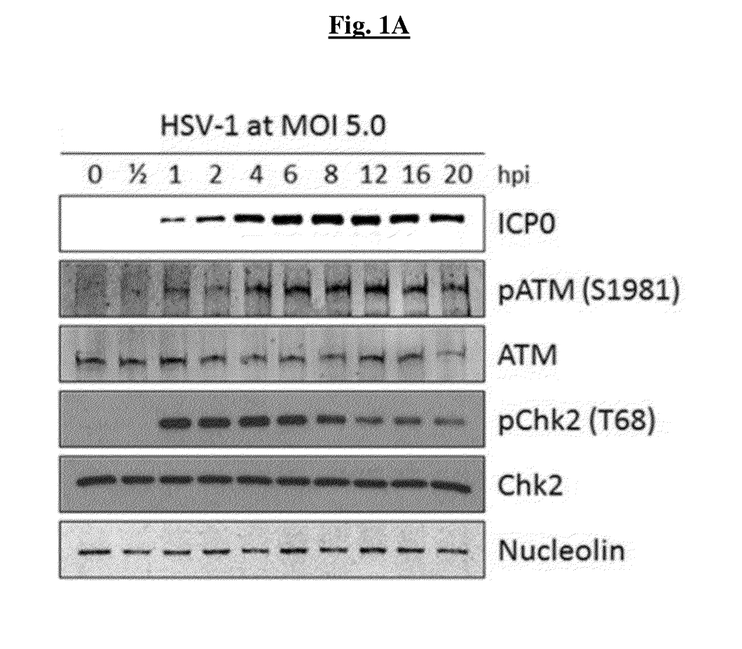

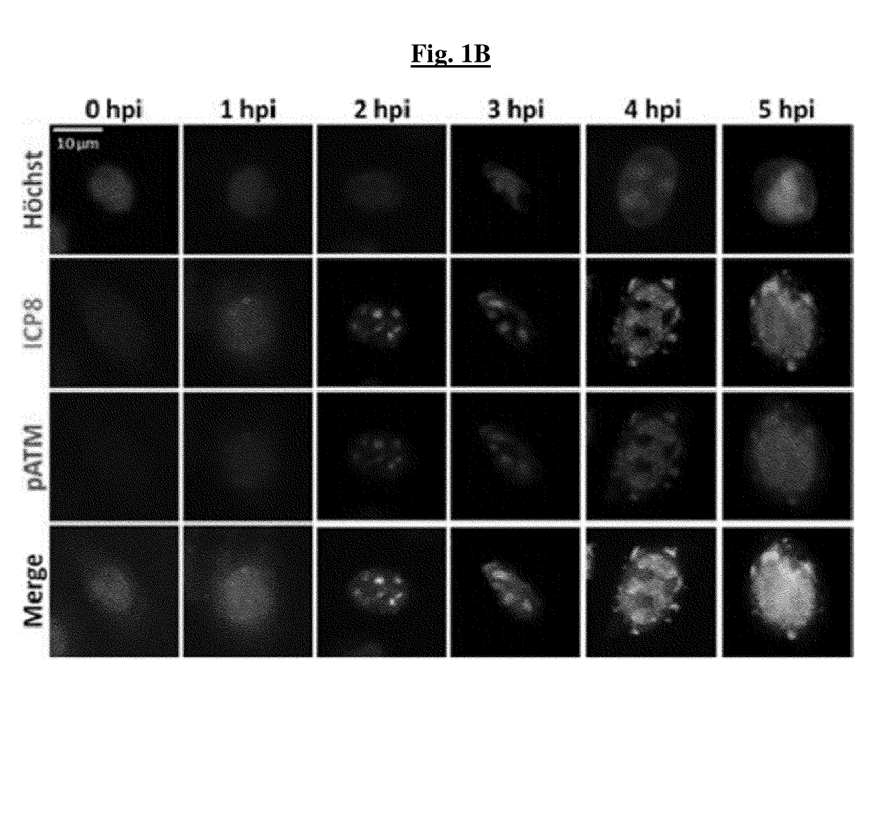

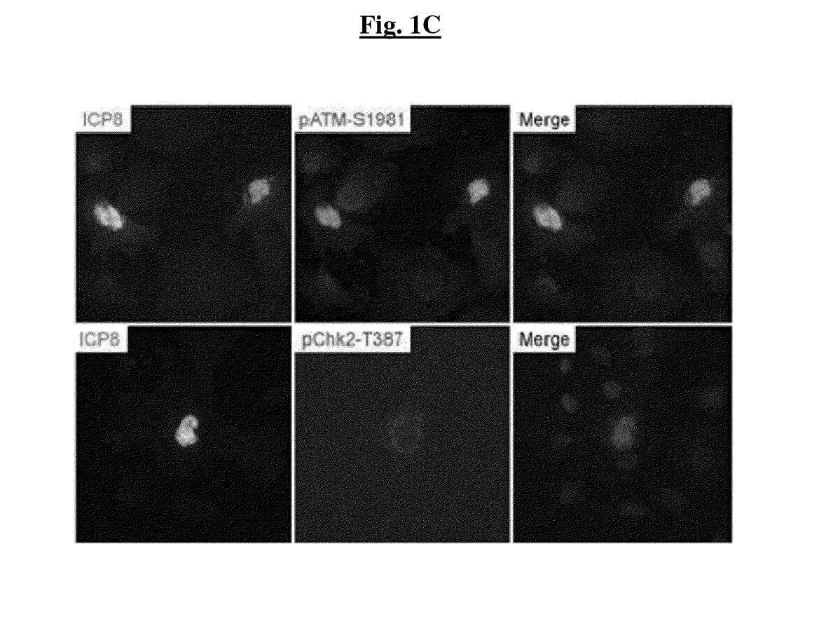

The induction of ATM activation by HSV-1 infection of human corneal epithelial cells was investigated. A time course of protein lysates from infected hTCEpi cells was analyzed by Western blot with antibodies against known phosphorylation targets of ATM—Ser1981 of ATM (autophosphorylation) and Thr68 of Chk2. ATM activity was observed as early as 1 hour post infection (hpi) and plateaued at a peak level between 4 and 6 hpi (FIG. 1A). Indirect immunofluorescence with pATM-specific antibodies demonstrated the expected pattern of ATM activation, which closely correlated with viral replication compartment dynamics (FIG. 1B). Diffuse weak pATM gradually concentrated in numerous small foci, which further coalesced to form larger areas, eventually taking over the entire nucleus by 5 hpi. The timing of maximum ATM activation detected by Western blot corresponded to the pan-nuclear stage of pATM staining.

### Example 2

### ATM Inhibition Suppresses HSV-1 Infection in Corneal Epithelial Cells

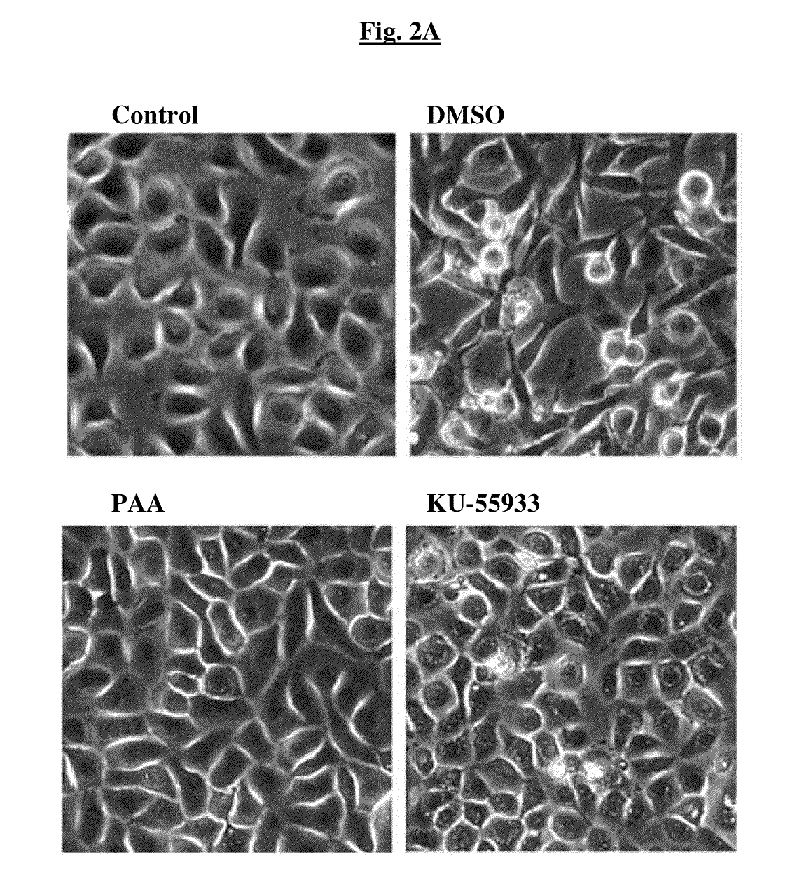

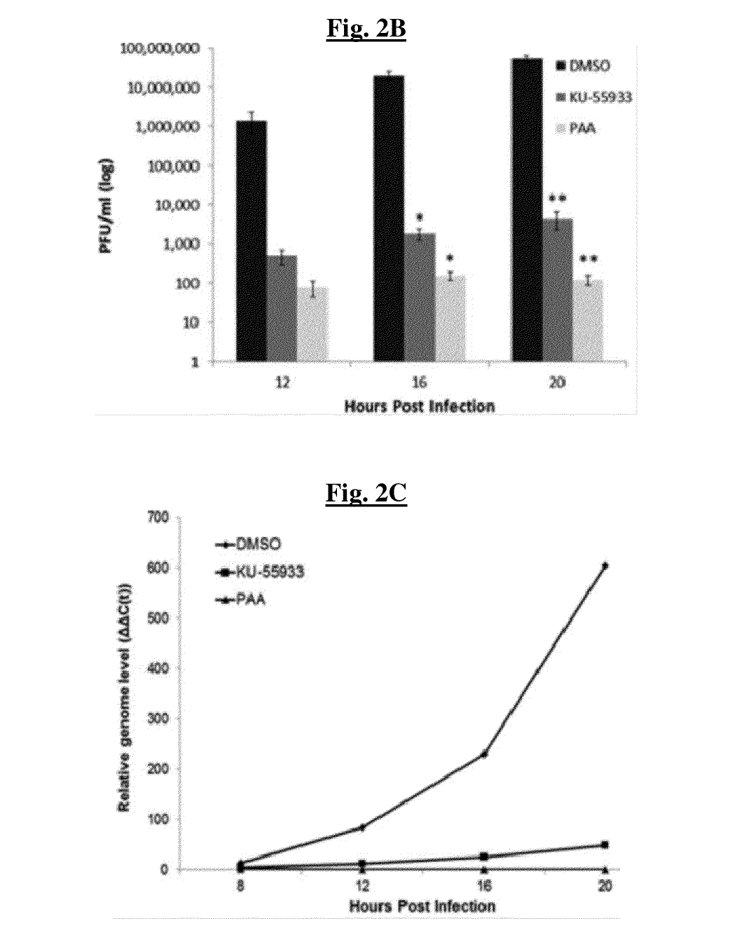

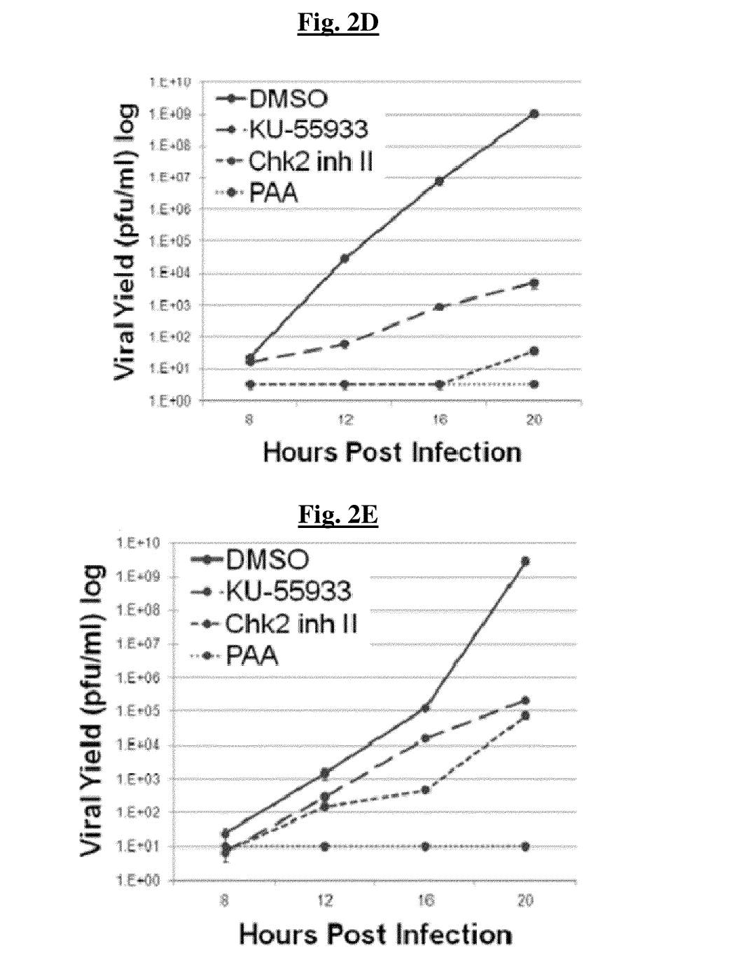

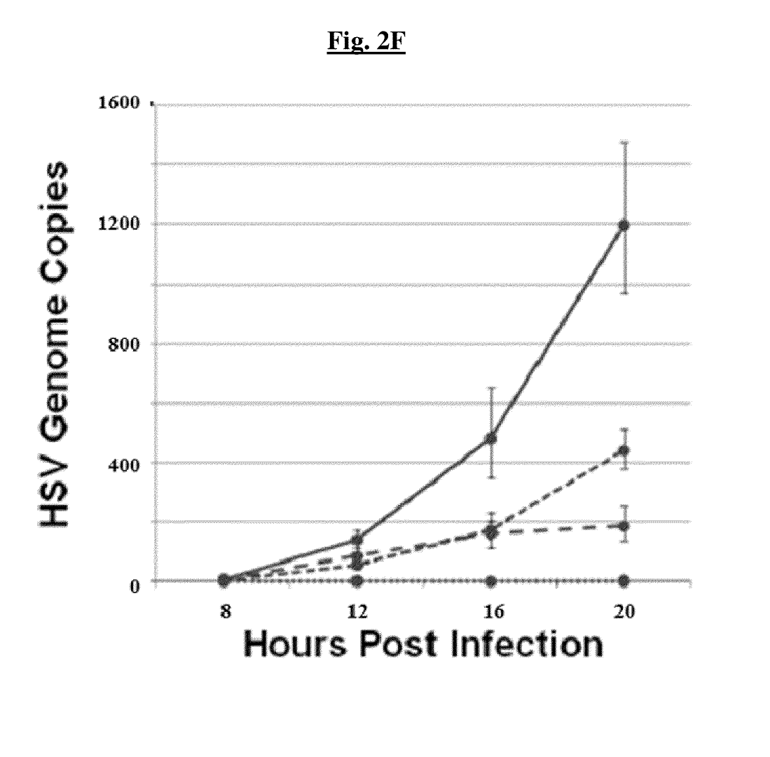

A highly specific small molecule inhibitor of ATM, KU-55933, was used to examine the effects of ATM inhibition on HSV-1 infection specifically in human corneal epithelial cells. KU-55933 prevented the cytopathic effect of HSV-1, which was otherwise pronounced in the mock treatment (FIG. 2A). Plaque assays revealed a potent inhibition (greater than 10.000-fold at 20 hpi) of infectious particle production associated with KU-55933 treatment of infected hTCEpi cells (FIG. 2B). The effect of ATM inhibition on viral genome replication was monitored by qPCR using primers against the viral genome. A sharp reduction in viral genome replication was observed throughout the course of infection in cells with inhibited ATM activity (FIG. 2C).

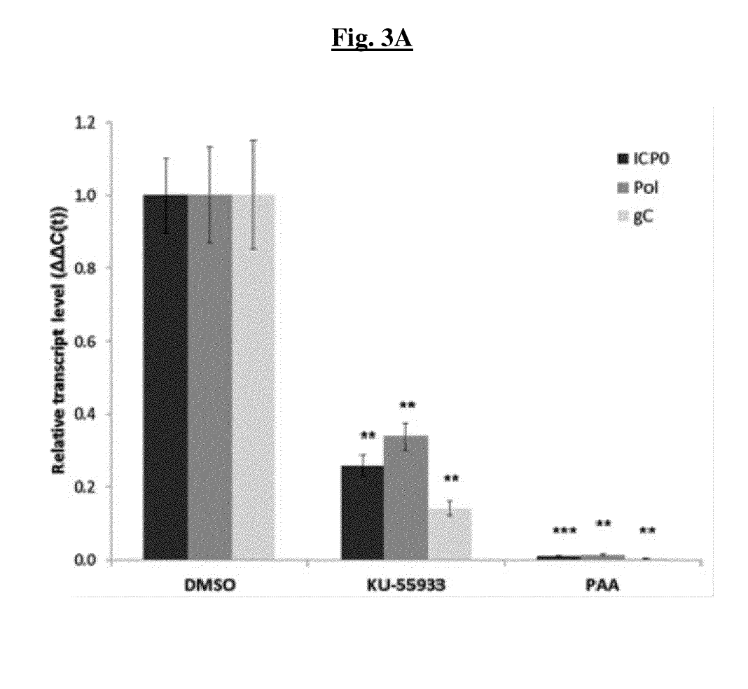

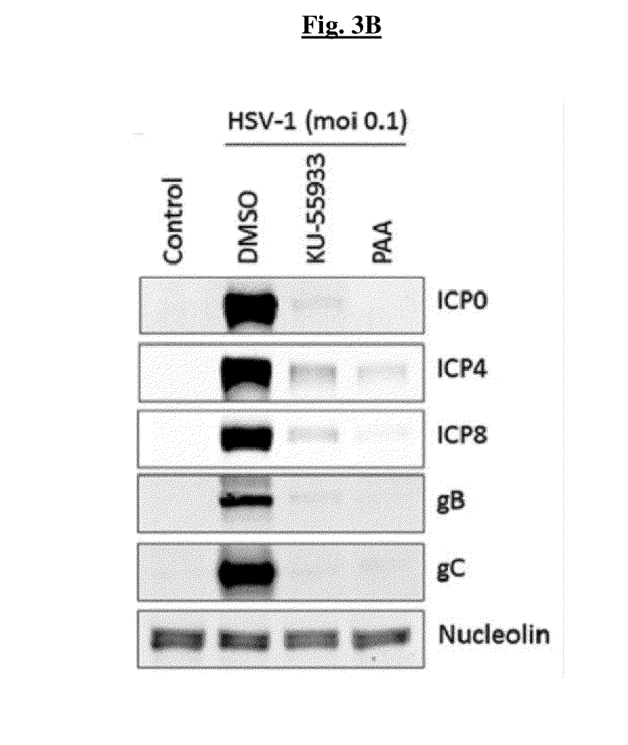

The inhibition of genome replication was associated with reduced accumulation of viral transcripts in the infected monolayers. Levels of viral transcripts from all three kinetic families—immediate early, early, and late—were reduced, as measured by qRT-PCR with primers against ICP0, DNA polymerase, and glycoprotein C, respectively (FIG. 3A). This reduction was accompanied by a pronounced decrease in the levels of viral proteins necessary for successful progression of the viral life cycle (FIG. 3B).

### Example 3

### ATM Inhibition Suppresses HSV-1 in Explanted Corneas

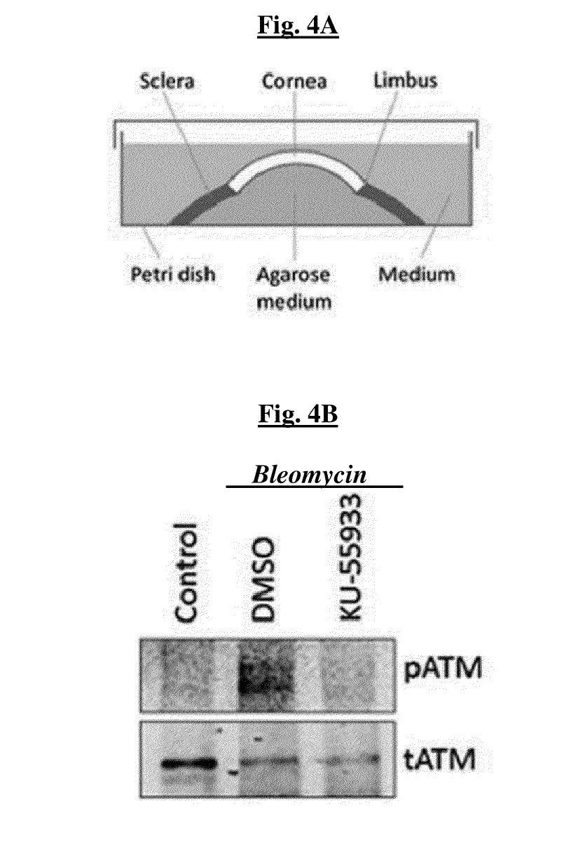

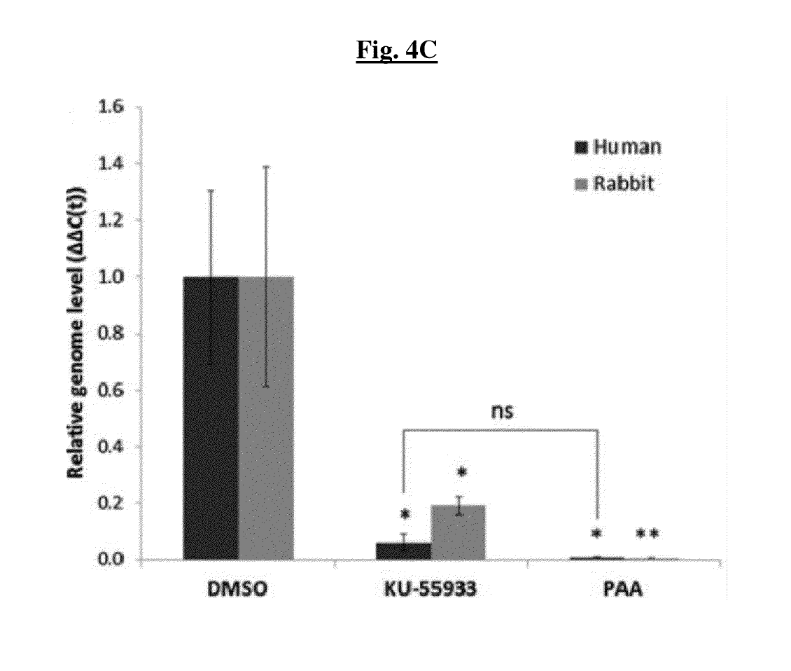

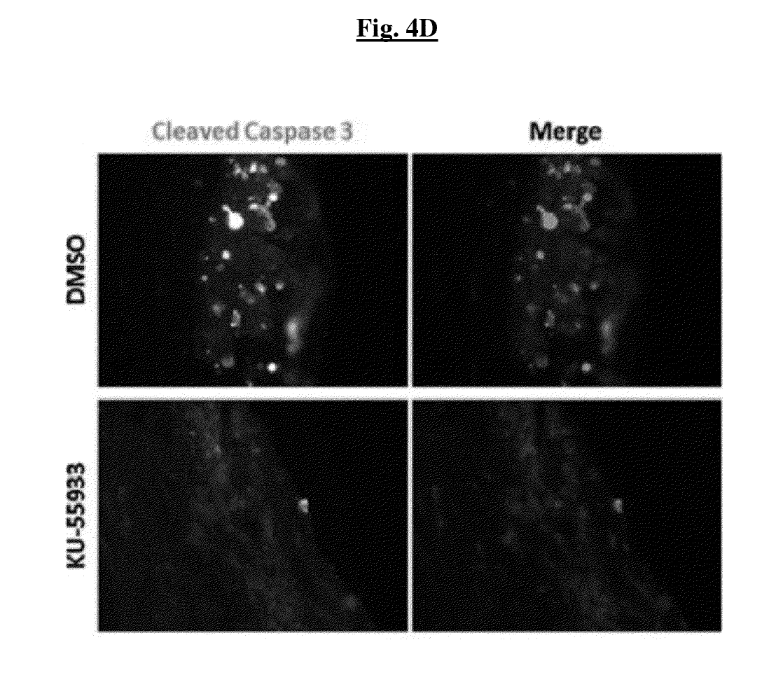

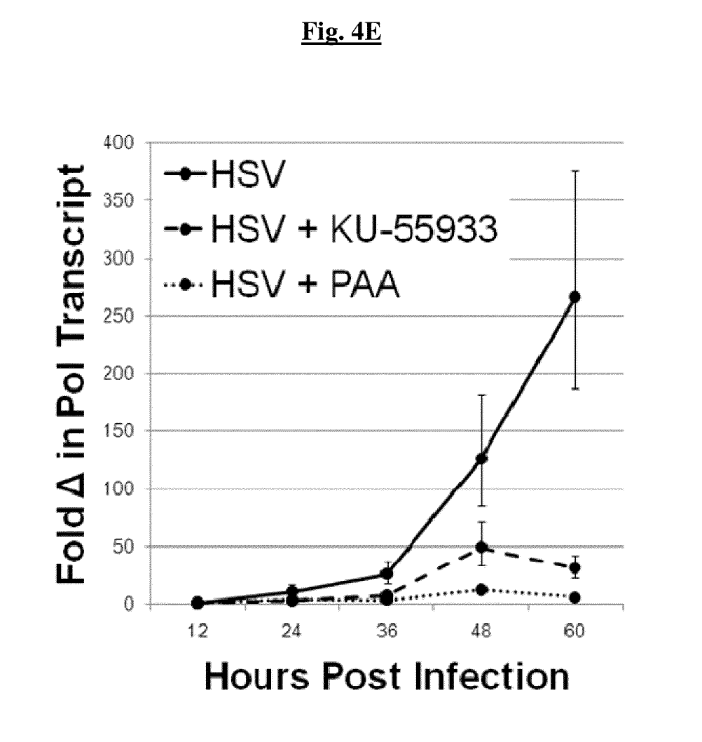

In order to study the antiviral effect of ATM inhibition in a more physiologically relevant model of epithelial herpes keratitis, an ex vivo model of corneal infection was developed. Intact corneoscleral buttons from humans and rabbits were infected and treated with KU-55933 in tissue culture (FIG. 4A). The bioavailability of KU-55933 in human corneal explants was evaluated by assessing its activity in the context of DNA damage induced by bleomycin, a known double strand break-inducing agent. Corneas damaged with bleomycin exhibited a high level of pATM, which was completely eliminated by pretreatment with KU-55933, demonstrating good penetration and activity of this inhibitor in the epithelial layers of an intact cornea (FIG. 4B). Consistent with the in vitro findings, viral genome replication in the epithelium of human and rabbit corneas was greatly reduced due to ATM inhibition (FIG. 4C). This effect was more pronounced in human corneas, likely due to the human specificity of the chemical structure of KU-55933. In addition, a reduction in cleaved caspase-3 staining, a marker of apoptosis, in the epithelium of ATM-inhibited corneas was observed as compared to mock-treated controls (FIG. 4D).

### Example 4

### KU-55933 Reduces Disease Severity in the Mouse Model of Herpes Keratitis

The in vitro (FIGS. 1A-1C, 2A-2F) and ex vivo (FIGS. 3A-3B) experiments demonstrate a pronounced reduction of viral replication in cells with inhibited ATM activity. Without wishing to be limited by any theory, while these data may relate well to epithelial keratitis, they do not necessarily predict an effect on stromal keratitis, a more severe form of herpetic corneal infection that is characterized by lymphocytic invasion of the stroma.

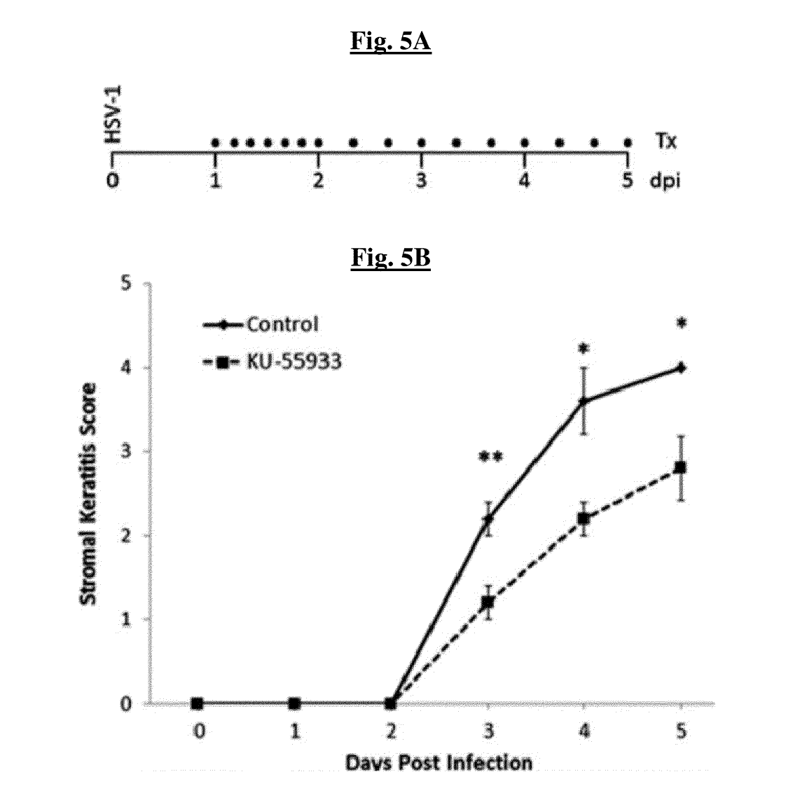

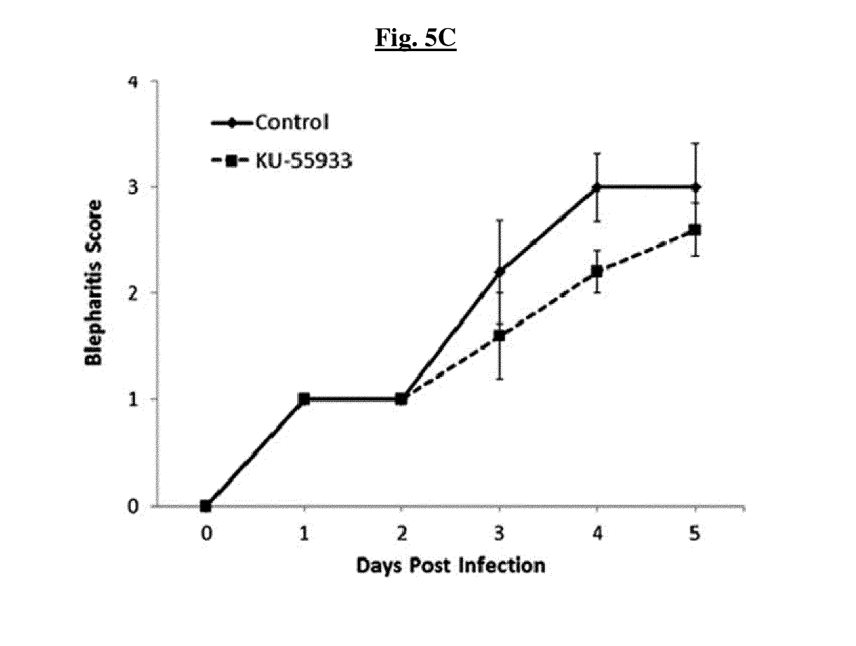

The mouse model of ocular HSV-1 infection was used to evaluate the effect of KU-55933 on the development of stromal disease. To increase the clinical relevance of the findings, mouse corneas were infected with McKrae strain, an ocular isolate of HSV-1, and infection was allowed to take place for a full day before initiation of treatments (FIG. 5A). KU-55933 treatments resulted in a notable and statistically significant reduction in stromal disease severity (FIG. 5B). For example, by day 5 postinfection, all of the control mice developed corneal perforation, while KU-55933-treated mice, on average, had only corneal opacity. Differences in the blepharitis score between the two groups were not statistically significant (FIG. 5C). The strong neurovirulence of the McKrae strain necessitated that the animals be euthanized before the resolution of disease.

### Example 5

### KU-55933 Exhibits Low Toxicity in Corneal Epithelium

The toxicity of ATM inhibition with KU-55933 in hTCEpi cells was assessed using the clonal survival assay, which revealed a roughly 70% survival of cells continuously treated with KU-55933 for 24 hours compared to the mock-treated controls (FIG. 6A). In addition, toxicity assessment was performed in explanted human corneas by fluorescein staining. No epithelial defects were detected after 30 hours of continuous treatment with KU-55933 (10 μM), while treatment with doxorubicin, a known proapoptotic agent, produced severe toxicity to the corneas (FIG. 6B). To assess the potential toxicity of prolonged KU-55933 treatment, fluorescein staining was similarly used on mouse corneas treated with 200 μM KU-55933 at the same schedule as outlined in FIG. 5A. Despite the frequent administration of KU-55933 for a total of 4 days, the corneas exhibited no epithelial ulceration or any other visually detectible abnormalities (FIG. 6C).

### Example 6

### Combination Treatments with KU-55933 and Acyclovir

The use of KU-55933 in combination with antiviral agents was investigated. A range of combined low concentrations of KU-55933 and acyclovir was used to treat infected hTCEpi monolayers. Quantitative PCR analysis of viral genome replication demonstrated an enhanced antiviral effect of the combined treatment as compared to the individual drugs alone. The addition of KU-55933 effectively shifted the acyclovir dose-response curve to the left (FIG. 7A). Acyclovir had a similar effect on the KU-55933 dose-response curve (FIG. 7B).

### Example 7

### Inhibition of Drug-Resistant HSV-1 by KU-55933

The antiviral activity of KU-55933 against a drug-resistant strain of HSV-1, dlsptk, was investigated. This strain harbors a mutation in the TK gene, which confers resistance against all antiviral agents that undergo activating phosphorylation catalyzed by this protein. dlsptk infection in hTCEpi cells was largely unresponsive to acyclovir treatment; however, KU-55933 was able to markedly suppress genome replication of the dlsptk strain (FIG. 8). The inhibitory effect of KU-55933 on dlsptk infection was as potent as its effect on KOS infection.

### Example 8

### Activation of Checkpoint Kinase 2 (Chk2) Is Critical for Herpes Simplex Virus Type 1 (HSV-1) Replication in Corneal Epithelium

### Materials and Methods

### Cells and Viruses

All cells were cultured at 37° C. and 5% CO₂, and supplemented with 100 U/ml penicillin and 100 μg/ml streptomycin. Human corneal epithelial cells immortalized with hTERT (hTCEpi; Bahassi et al., 2008, Oncogene 27:3977-3985) were grown in complete KGM-2 medium. Human corneal epithelial cells immortalized with SV40 large T antigen (HCE; Araski-Sasaki et al., 1995, Invest. Ophthalmol. Visual Sci. 36:614-621), as well as African green monkey kidney fibroblasts (CV-1; Jensen et al., 1964, Proc. Natl. Acad. Sci. USA 52:53-59), were grown in DMEM medium supplemented with 10% FBS. KOS strain (Smith, Proc. Soc. Exp. Biol. Med. Soc. Exp. Biol. Med. 115:814-816) of HSV-1 was used in all infections. All viral stocks were titered on CV-1 monolayers.

Tetracyline-inducible Chk2 knockdown cell line was derived by lentivirally transducing HCE cells with a construct harboring shRNA sequence against the Chk2 transcript. The Chk2 shRNA sequence was acquired from Sigma (NM_{—}007194.2-1299s1c1) and targets the following region: 5′-CGCCGTCCTTTGAATAACAAT-3′ (SEQ ID NO:1). Lentiviral particles were produced in 293T packaging cells (Dull et al., 1998, J. Virol. 72:8463-8471). HCE cells were selected with neomycin after transduction and knockdown induction was verified by Western blot. Chk2 was optimally knocked down after a 72-hour treatment with doxycycline (0.25 μg/ml).

### Infection and Treatments of Cultured Cells

Cells were grown in 6-well plates and used in experiments at ˜80% confluence. Drug treatments were administered 45 min prior to infection and continued for the entire duration of each experiment. Unless indicated otherwise, Chk2 inhibitor II (>98% purity by HPLC) was used at 10 μM final concentration, and phosphonoacetic acid (PAA) at 400 μg/ml (both from Sigma-Aldrich, St. Louis, Mo.). Chk2 inhibitor II was dissolved in DMSO such that the final concentration of DMSO in both Chk2 inhibitor II and mock treatment was 0.1%. Infections with KOS strain of HSV-1 were carried out in 6-well plates in a 200 μl inoculum volume at 37° C. for 1 hour with intermittent rocking. The cells were then thoroughly rinsed and overlaid with fresh medium.

### Corneal Explant Model

Human corneas were obtained from the Lions Eye Bank of Delaware Valley. Rabbit corneas were excised from intact fresh eyeballs of young (8-12 weeks) albino rabbits (Pel-Freez Biologicals, Rogers, Ark.). The protocol (Alekseev et al., 2012, J. Vis. Exp. e3631) for ex vivo corneal culture and infection was followed, and treatment was administered immediately after infection. Briefly, corneoscleral buttons were excised and rinsed in PBS containing 200 U/ml penicillin and 200 μg/ml streptomycin. The endothelial concavity was filled with culture medium containing 1% low melting temperature agarose. The corneas were cultured epithelial side up in MEM medium supplemented with non-essential amino acids (1×), 2 mM L-glutamate, 200 U/ml penicillin, and 200 μg/ml streptomycin. The next day, they were infected with 1×10⁴ PFU/cornea of strain KOS HSV-1 for 1 hour, rinsed, and overlaid with fresh medium. Drug treatments were administered at the same concentrations as for cultured cells. The epithelial cell layer was collected by scraping the corneas for isolation of total DNA. For immunohistochemistry studies, corneas were flash-frozen in OCT compound, sectioned, and immunostained using standard protocols.

### Viral Replication

Total DNA from infected cells and corneas was isolated using the DNeasy Blood & Tissue Kit (QIAGEN, Hilden, Germany). Real time quantitative PCR was performed with SYBR Green (Bio-Rad, Hercules, Calif.). Target primers for UL30 (DNA polymerase catalytic subunit) and reference primers for GAPDH were used to measure viral genome abundance. Primer sequences were based on the KOS genome (accession #JQ673480.1). UL30 primers (Kim et al., 2005, Cell. Immunol. 238:76-86) (Fwd: AGAGGGACATCCAGGACTTTGT, SEQ ID NO:2; Rev: CAGGCGCTTGTTGGTGTAC, SEQ ID NO:3) produce a 74 by amplicon, and GAPDH primers (Berkovich et al., 2007, Nat. Cell. Biol. 9:683-690) (Fwd: GCTTGCCCTGTCCAGTTAAT, SEQ ID NO:4; Rev: TAGCTCAGCTGCACCCTTTA, SEQ ID NO:5) produce a 101 by amplicon. All real time PCR data were processed using the Pfaffl method (Pfaffl, 2001, Nucleic Acids Res. 29:e45), which yields relative template levels via this equation:

$$ΔΔ C ( t ) = \frac{{E}_{target}^{{C ( t )}_{control} - {C ( t )}_{sample}}}{{E}_{reference}^{{C ( t )}_{control} - {C ( t )}_{sample}}}$$

Primer efficiencies (E) were calculated for both primer pairs. Melt peaks were examined for every reaction in every experiment, and reactions with aberrant melt peaks were excluded from calculations.

### Immunohistochemistry

Corneas were flash-frozen in OCT compound, sectioned at 10 micron thickness, dried, fixed in 3% paraformaldehyde/2% sucrose solution for 10 min, and permeabilized with 0.5% Triton X-100 for 5 min. Indirect immunofluorescence was performed with primary antibodies against cleaved caspase-3 (rabbit polyclonal; Cell Signaling, Danvers, Mass.). Nuclei were counterstained with 10 mg/ml Hochst 33258.

### Western Blot

Standard protocol was followed for Western blot analysis. Cell lysates were collected in 200 μl of Laemmli buffer, vortexed, and boiled at 95° C. for 5 min. Protein concentrations were measured by reducing agent-compatible BCA assay. SDS-PAGE was followed by transfer onto a PVDF membrane, which was then blocked in 5% BSA. Primary antibodies against the following proteins were used: nucleolin (mouse monoclonal; Santa Cruz Biotechnology, Santa Cruz, Calif.), ATM and pATM S1981 (rabbit polyclonal and mouse monoclonal, respectively; Rockland, Glibertsville, Pa.), Chk2 and pChk2 T68 (rabbit polyclonal and mouse monoclonal, respectively; Cell Signaling, Danvers, Mass.). Blots were stained with secondary antibodies and visualized with the Odyssey near-infrared system (LI-COR, Lincoln, Nebr.).

### Statistical Analysis

Statistical significance was determined using Student's t-test and is indicated with ns (p>0.05), * (p<0.05), ** (p<0.01), or *** (p<0.001).

Experimental results are now exemplified.

### Inhibition of Chk2 Suppresses HSV-1 Replication in Human Corneal Epithelial Cells

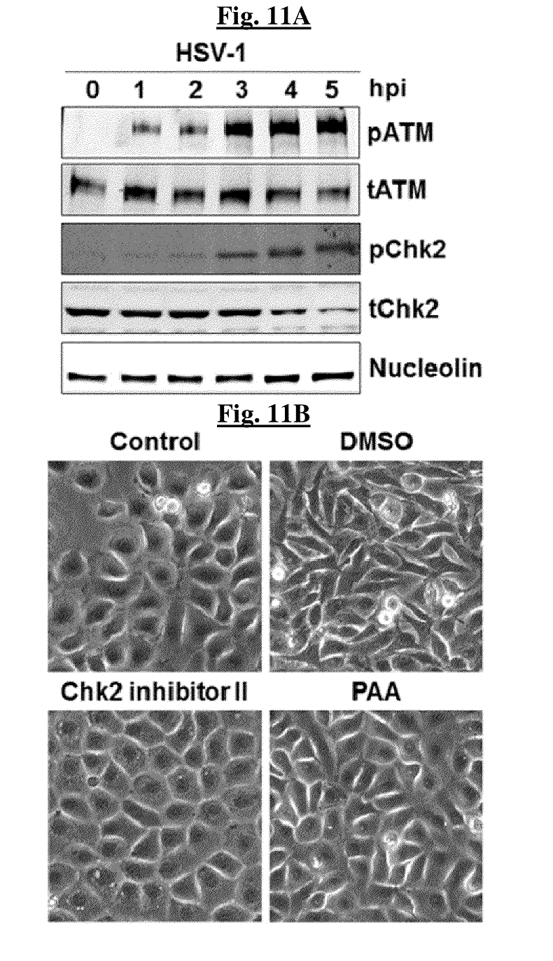

The activating autophosphorylation of ATM (Ser 1981) and the subsequent activating phosphorylation of Chk2 (Thr 68) are detected within the first hour of HSV-1 infection (FIG. 11A). Human colorectal carcinoma cells (HCT116) deficient in Chk2 expression are impaired in their ability to support productive HSV-1 infection compared to Chk2-expressing controls. In order to address this phenotype in non-tumorigenic cells, two human corneal epithelial cell lines—hTCEpi and HCE, which are known to be contact-inhibited and are derived from healthy corneas, were used. These cell lines were also chosen based on their different immortalization methods (hTERT and SV40 large T antigen, respectively) to exclude the possibility of immortalization-specific results.

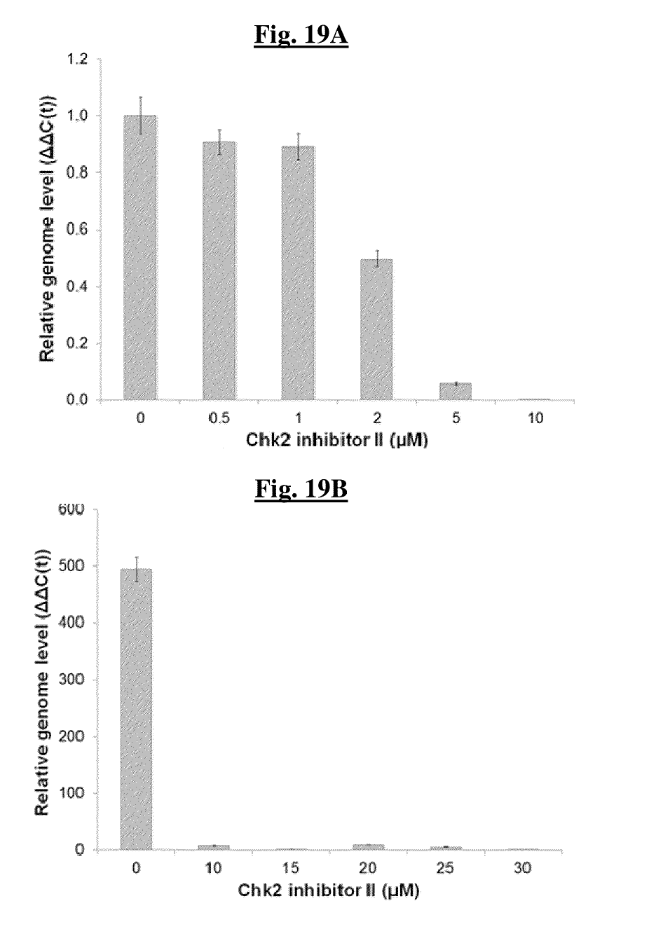

Sub-confluent cells were infected with HSV-1 at a relatively low multiplicity of infection (MOI 0.1) to imitate the physiological condition, and a highly specific small molecule inhibitor of Chk2, Chk2 inhibitor II, was used to assess the significance of this kinase during infection. Dose-optimization in hTCEpi cells was performed, which confirmed the 10 μM concentration (FIG. 19A). Treatment with this inhibitor almost completely eliminated the cytopathic effect (CPE) associated with HSV-1. CPE reduction was pronounced even past 20 hpi (FIG. 11B), a time point at which these cells undergo at least three rounds of re-infection.

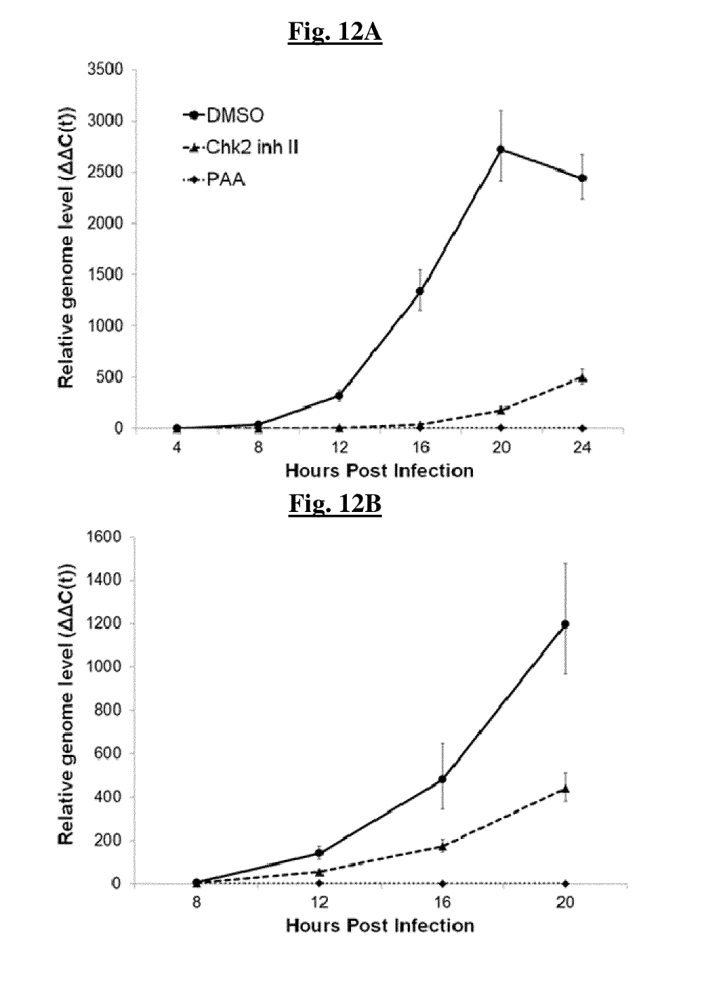

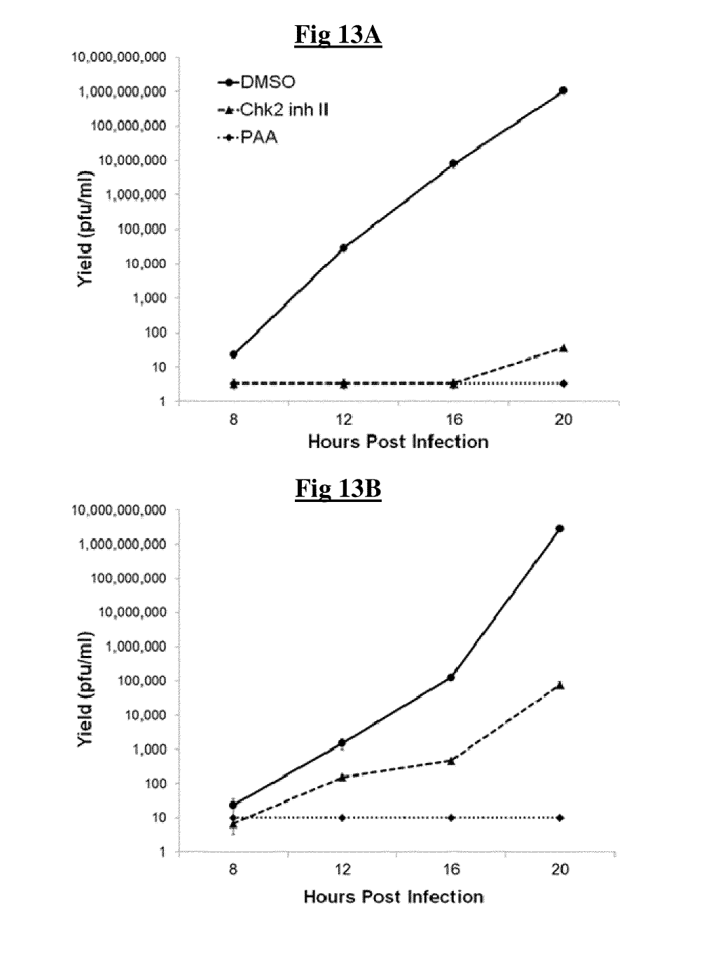

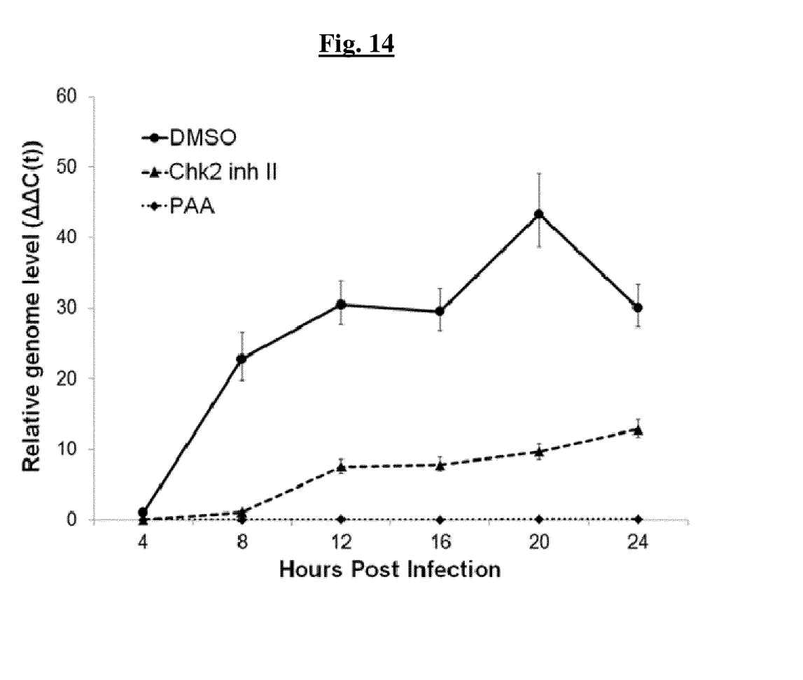

To obtain a quantitative measure of the antiviral effect of Chk2 inhibitor II, a qPCR assay was performed to detect viral genomes in the treated monolayers. Inhibition of Chk2 profoundly reduced viral replication in both cell types (FIGS. 12A-12B). Accordingly, this inhibitory effect was paralleled by a reduction in the generation of infectious viral particles in treated cells compared to controls, as measured by plaque assay (FIGS. 13A-13B). To test the antiviral potency of Chk2 inhibitor II in a setting of heavy HSV-1 infection, hTCEpi cells that had been infected at MOI 5, a viral load 50-fold higher than that used earlier, were treated. qPCR measurement of viral genome levels revealed a reduced yet still substantial decrease in replication associated with Chk2 inhibition (FIG. 14).

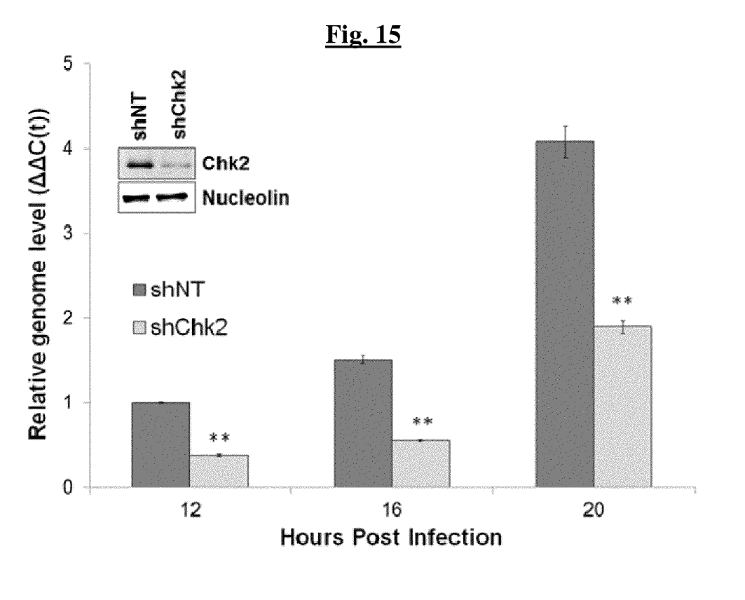

In order to confirm the antiviral effect of the inhibitor, interference with Chk2 activity was implemented using RNAi-mediated gene knockdown. Stable depletion of Chk2 in normal corneal epithelial cells was not possible due to its toxic consequences. To circumvent this, HCE cells were used to derive stable cell lines harboring tetracycline-inducible shRNA against Chk2 or non-targeting shRNA control. Chk2 knockdown was induced with doxycycline for 72 hr prior to infection with HSV-1, and genome replication was measured by qPCR. Chk2 protein levels were assessed by Western blot using lysates collected at the time of infection (FIG. 15 inset). Chk2 knockdown had an inhibitory effect on viral infection in HCE cells (FIG. 15), albeit not as pronounced as the effect of Chk2 inhibitor II. This discrepancy is most likely due to the residual Chk2 kinase that could not be eliminated in the system, since densitometry measurements show incomplete knockdown (81.7%). Without wishing to be limited by any theory, it is also possible that the inhibitor may exert off-target effects that contribute to reduced viral replication. Nevertheless, this result agrees with our inhibitor data and confirms that the antiviral activity of Chk2 inhibitor II, at least to a large extent, is achieved through specific inhibition of the Chk2 kinase.

### Inhibition of Chk2 Suppresses HSV-1 Replication in Explanted Human and Rabbit Corneas

In order to extend the in vitro findings to a physiologically relevant model, ex vivo corneal HSV-1 infection was performed. Human and rabbit corneoscleral buttons were maintained in organotypic tissue culture and infected with HSV-1 in the presence of Chk2 inhibitor II. 10 μM drug concentration was used based on additional dose optimization carried out in explanted human corneas (FIG. 19B). qPCR measurement of viral genome levels at 48 hpi demonstrated that corneas treated with the inhibitor did not support productive infection, as compared to mock-treated controls (FIGS. 6A-6C). There was no statistical significance between viral genome levels in the Chk2 inhibitor II-treated human corneas and positive controls treated with PAA. HSV-1 inhibition was slightly less potent in rabbit than in human corneas, which may be explained by the specificity of the inhibitor for the human enzyme.

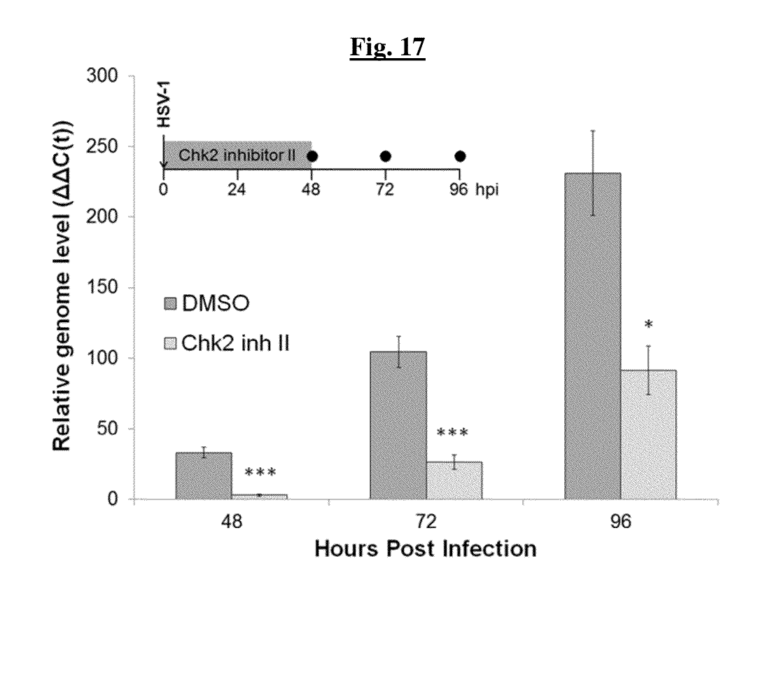

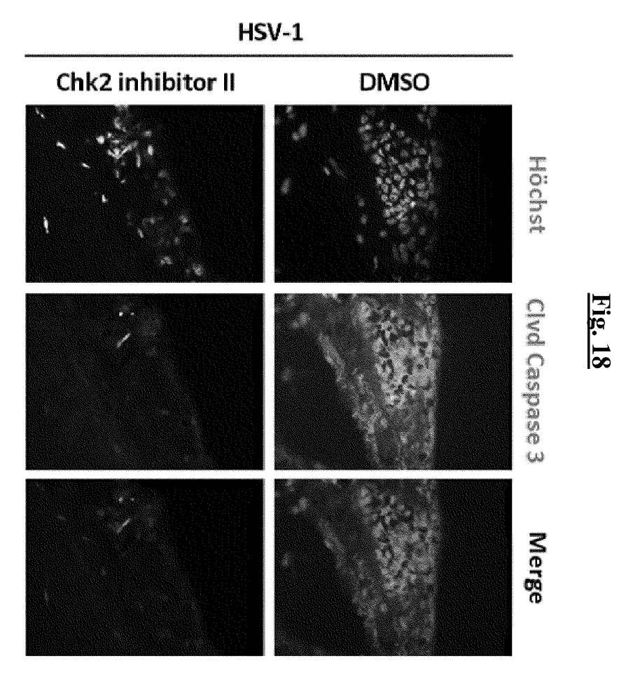

In light of these findings, the long term effects of Chk2 inhibition in the explant model were explored. To this end, rabbit corneas were infected and maintained in culture with uninterrupted treatment with Chk2 inhibitor II for two days. At this point, the drug was removed from the medium, and all corneas were cultured in inhibitor-free medium for two more days, during which time epithelial DNA samples were collected (FIG. 17 inset). qPCR analysis revealed a lasting effect of Chk2 inhibition that was maintained as late as 96 hpi (latest time point tested) (FIG. 17). HSV-1 seemed to resume normal growth following the removal of inhibitor, indicating that Chk2 inhibition suppresses viral replication, but does not eliminate the infected cells.

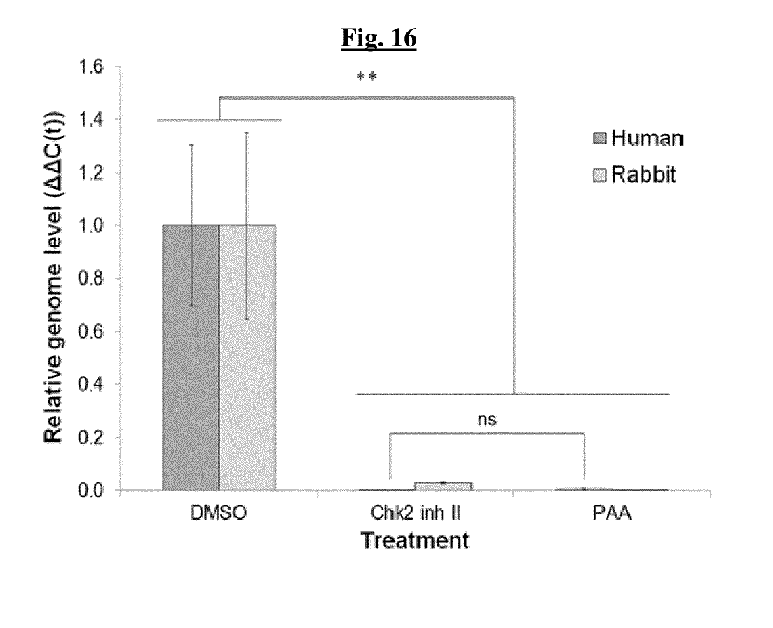

The effect of Chk2 inhibition on the overall corneal health during infection was assessed. Explanted human corneas were infected with HSV-1 and treated with Chk2 inhibitor II or mock (DMSO) (FIG. 16). At 48 hpi, corneas were analyzed by immunohistochemistry with antibodies against cleaved caspase-3, a common marker of apoptosis. Mock-treated corneas developed notable limbal apoptosis in response to HSV-1 infection; however, this was abrogated in corneas treated with Chk2 inhibitor II (FIG. 18).

### Example 9

### HSV-1 Hijacks the Host DNA Damage Response through ICP4-Mediated Activation of ATM

### Materials and Methods

### Cells

All cells were cultured at 37° C. and 5% CO₂, and supplemented with 100 U/ml penicillin and 100 μg/ml streptomycin. hTCEpi human corneal epithelial cells were cultured in KGM-2 (Lonza, Basel, Switzerland). HCE human corneal epithelial cells were cultured in DMEM/F-12 supplemented with 10% FBS. EPC2 human esophageal epithelial cells were cultured in KSFM (Carlsbad, Calif.). OKF6 human oral epithelial cells were cultured in KSFM. ES cells, which are CV-1 cells stably expressing HSV-1 ICP4 protein, were cultured in DMEM supplemented with 10% FBS. HEK293 human embryonic kidney epithelial cells, HeLa human cervical adenocarcinoma cells, U2OS human osteosarcoma cells, H1299 human lung carcinoma cells, and SH-SY5Y human neuroblastoma cells were all obtained from American Type Culture Collection and cultured in DMEM supplemented with 10% FBS.

### Viruses

All HSV-1 virus stocks were prepared and titered on CV-1 monolayers and stored at −80° C. 7134 strain was an ICP0 double deletion mutant. tsB7 strain was a temperature sensitive nuclear entry mutant. d120 strain was an ICP4 double deletion mutant.

### Treatments

Transfection of plasmids was done with GenDrill (BamaGen, Gaithersburg, Md.), whereas transfection of BACs was accomplished with Lipofectamine transfection reagent (Invitrogen, Carlsbad, Calif.). All transfections followed standard protocols and manufacturer's instructions. Medium was changed at 6 hour post transfection.

### Western Blot

Standard protocol was followed for Western blot analysis. Cell lysates were collected in Laemmli buffer, vortexed, and boiled at 95° C. for 5 min. Protein concentrations were measured by reducing agent-compatible BCA assay. SDS-PAGE was followed by transfer onto a PVDF membrane, which was then blocked in 5% BSA. Primary antibody staining was performed overnight and blots were visualized on film or with the Odyssey near-infrared system (LI-COR, Lincoln, Nebr.). Primary antibodies against the following proteins were used: ICP0 (Virusys Corporation, Taneytown, Md.), ICP4, PML, and nucleolin (Santa Cruz Biotechnology, Santa Cruz, Calif.), ICP8, glycoproteins B and C, ATM and pATM-Ser1981 (Rockland, Gilbertsville, Pa.), Chk2 and pChk2-Thr68 (Cell Signaling, Danvers, Mass.).

### Immunofluorescence

Cells were grown on cover slips, treated as indicated, fixed in 3% paraformaldehyde/2% sucrose solution for 10 min, and permeabilized with 0.5% Triton X-100 for 5 min. Indirect immunofluorescence was performed with primary antibodies overnight followed by secondary antibody staining for 2 hours. Primary antibodies were the same as those used for Western blotting. Nuclei were counterstained with 10 mg/ml of Hochst 33258.

qRT-PCR

Total DNA was isolated from infected cells using the DNeasy Blood & Tissue Kit (QIAGEN, Hilden, Germany). Real time quantitative PCR was performed with SYBR Green (Bio-Rad, Hercules, Calif.). Target primers for UL30 (DNA polymerase catalytic subunit) and reference primers for GAPDH were used to measure genome replication. Primer sequences were provided in Alekseev et al., 2014, Invest. Ophthalm. Vis. Sci. 55:706-715). Real time PCR data were processed using the ΔΔC(t) method.

### Cells and Treatments

hTCEpi, EPC2, and OKF6 are normal human epithelial cells from cornea, esophagus, and palate, respectively. HEK293 are human embryonic kidney epithelial cells. Cells were treated with tissue culture grade KU-55933 (10 μM), cycloheximide (5 μg/ml), phosphonoacetic acid (400 μg/ml), and H₂O₂ (150 μM). Viral particles were pre-treated with UV light at 0.2 J/cm².

### Infections

Cultured cells were infected by applying the desired viral load in an inoculum volume equal to 10% of the normal volume of growth medium. During infection, cells were maintained at 37° C. and 5% CO₂ and rocked every 10-15 min for 1 hour. Cells were then rinsed thoroughly with PBS and overlaid with fresh medium. For synchronized infections, virus was allowed to adsorb to cells while rocking at 4° C. After 1 hour, cells were transferred to 37° C. and 5% CO2 to initiate synchronized infection.

### Comet Assay

Standard comet assay protocol (Olive et al., 2006, Nature Protocols 1:23-29) was followed. Briefly, treated cells were suspended by trypsinization, mixed with 1% low-melting temperature agarose, and pipetted onto agarose covered slides, which were submerged in alkaline lysis solution (1.2 M NaCl, 100 mM Na₂EDTA, 0.1% sarkosyl, 0.26 M NaOH (pH>13)) for 18-20 hours at 4° C. in the dark. Alkaline rinse solution (0.03 M NaOH, 2 mM Na₂EDTA (pH12.3)) was used to remove traces of salt and detergent. Slides were electrophoresed in fresh rinse solution for 25 min at 0.6 V/cm and stained with 2.5 μg/ml propidium iodide for 20 min. Individual comet images were obtained using an inverted fluorescence microscope (Leica DM-IRB, Wetzlar, Germany) and analyzed with CometScore® software (TriTek, Sumerduck, Va.).

### Statistical Analysis

Statistical significance was determined using Student's two-tailed t-test and is indicated with ns (p>0.05), * (p<0.05), ** (p<0.01), or *** (p<0.001).

The present study investigates the causative mechanisms of ATM activation during HSV-1 infection. As demonstrated herein, ATM is activated independently of damaged DNA and in a manner dependent on the viral immediate early gene product ICP4. The presence of the viral genome in the nucleus is also necessary for ATM activation, which suggests a genome-ICP4 interaction that may underlie this critical step in the viral life cycle. Investigations of the kinetics of this phenomenon point to the existence of a very early ATM-dependent step in the lytic cycle of HSV-1. Experimental results are illustrated below.

### HSV-1 Activates ATM in the Absence of DNA Damage

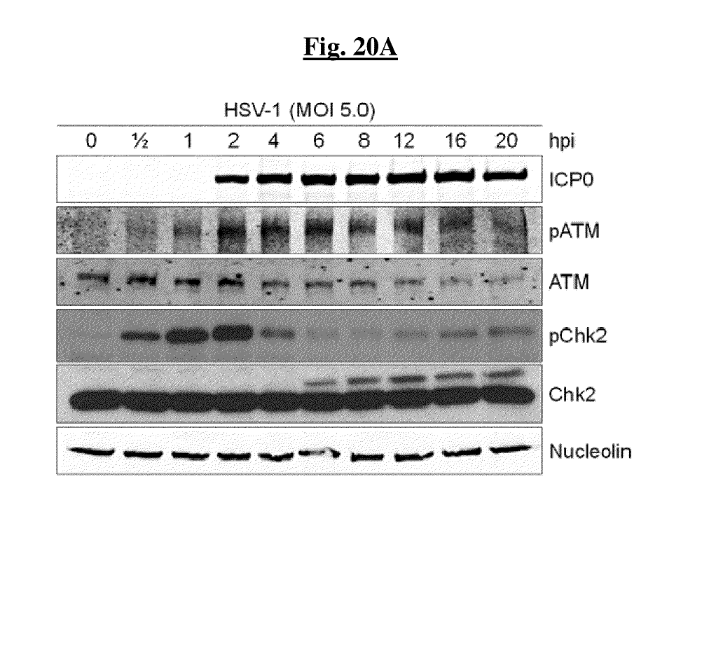

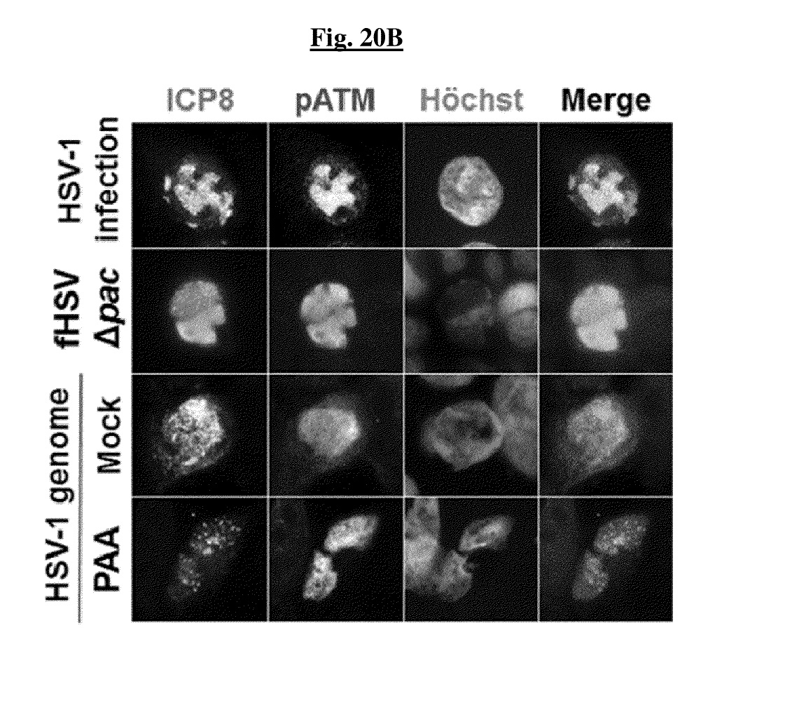

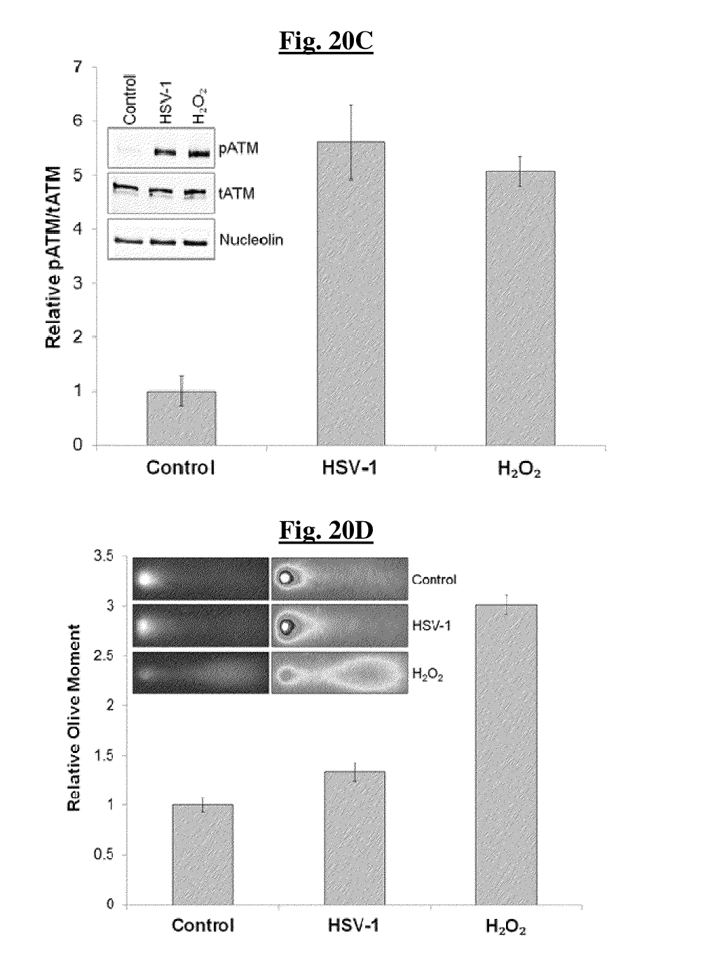

HSV-1 infection elicits robust activation of ATM in the host cell (FIG. 20A). The incoming HSV-1 genome is a linear double stranded DNA molecule that contains single-stranded nicks and gaps in the sugar-phosphate backbone. These features, along with the ends of the linear genome, may be detected as DNA damage and trigger ATM activation.

fHSVΔpac, a bacterial artificial chromosome (BAC) that contains the full HSV-1 genome with a deletion of both pac sequences, was used herein. Transfection of fHSVΔpac into HEK293 cells successfully activated ATM despite the absence of linear ends and single-stranded damage. Activated ATM colocalized to the viral replication compartments, resulting in a pattern similar to that seen in HSV-1 infection (FIG. 20B, top panels). Thus, this result excludes the genome integrity defects and linear ends of the HSV-1 genome as being required for ATM activation.

Nuclear injection of the viral genome and the initiation of stressful events, such as chromatin remodeling, induction of apoptosis, and dysregulation of repair pathways, could spotentially activate ATM through the induction of cellular DNA damage. To address this hypothesis, the amount of nuclear DNA damage induced in response to HSV-1 infection or hydrogen peroxide treatment was compared, under conditions in which these two stimuli produce equivalent levels of ATM activation (FIG. 20C). OKF6 cells were processed by comet assay for detection of nuclear DNA damage. Olive moment measurements revealed far greater nuclear DNA fragmentation in the peroxide-treated cells compared to HSV-1-infected cells, whose DNA damage levels were similar to those of untreated controls (FIG. 20D). Therefore, the level of ATM activation in HSV-1 infection is disproportional to the amount of DNA damage in the host cell.

Taken together, these experiments provide evidence that ATM is activated during HSV-1 infection independently of the presence of DNA damage, whether in the host or in the viral genome. Without wishing to be limited by any theory, these data suggest a DNA damage-independent mechanism that may be used by the virus to activate ATM.

### Full ATM Activation by HSV-1 Requires Nuclear Entry of the Genome and De Novo Protein Synthesis

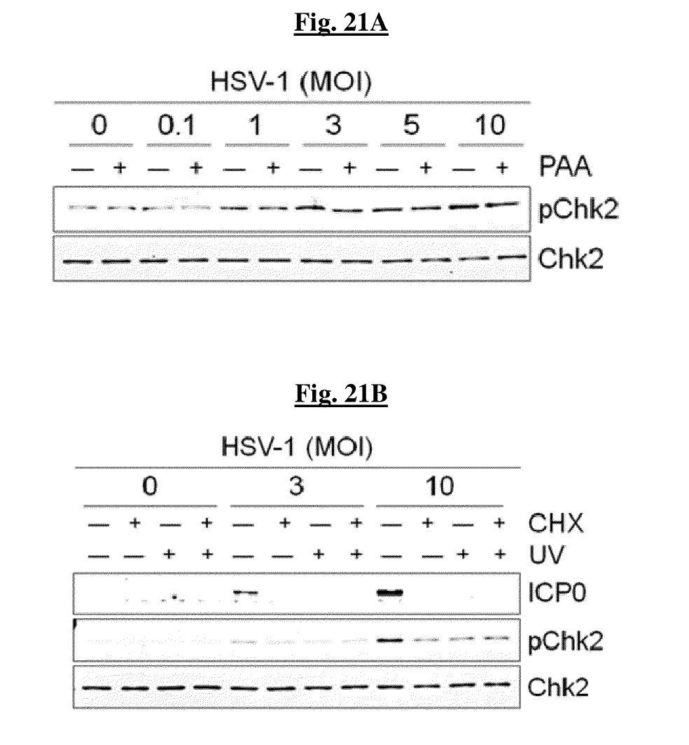

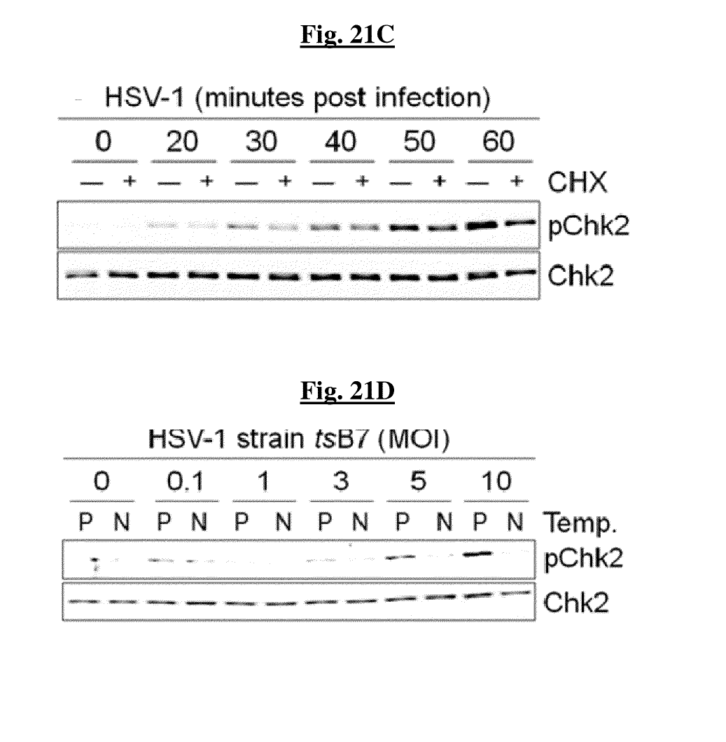

The process of viral genome replication generates complex concatameric and branched DNA structures and experiences replication fork collapse, and ATM activation may occur in a replication-dependent manner. However, infection of hTCEpi cells in the presence of a viral DNA polymerase inhibitor, phosphonoacetic acid (PAA), had no effect on ATM activation (FIG. 21A). This was assayed by Western blot staining for pChk2, a direct target of ATM, whose phosphorylation level is a surrogate measure of ATM activity. To confirm this result, purified HSV-1 genome was transfected into HEK293 cells that were cultured in the presence or absence of PAA. Untreated cells exhibited strong ATM activation that co-localized to the replication compartments. In line with the Western blot data, PAA-treated cells were not hindered in ATM activation, despite the absence of proper replication compartments (FIG. 20B, bottom panels). Together, these experiments demonstrate that ATM activation occurs in HSV-1-infected cells independently of the viral replication processes.

Post-translational modifications of host factors by viral proteins are diverse and well documented for many viruses, including HSV-1. To address the possibility that a specific virally encoded protein is involved in ATM activation, hTCEpi were infected cells in the presence or absence of cycloheximide (CHX), an inhibitor of the ribosome. Western blot staining for pChk2 revealed a partial inhibitory effect of CHX on ATM activation. This partial inhibition was highly consistent and replicable and held true for all MOIs tested (FIG. 21B). To rule out the possibility that the CHX effect is simply due to the inhibition of a host protein, viral particles were pre-treated with ultraviolet (UV) light to specifically inhibit viral protein synthesis. Infection of hTCEpi cells with UV-pretreated virus produced the same partial inhibitory effect on ATM activation (FIG. 21B), demonstrating the involvement of a viral protein. Combination treatment with CHX and UV produced no additional reduction of ATM activation, further supporting the activating role of a viral protein. Since total inhibition of de novo viral protein synthesis produced only a partial reduction of ATM activation, the responsible protein may have a dual source—from de novo synthesis and from the tegument. Thus, CHX and UV would only inhibit ATM activation achieved through the de novo synthesized protein, but not prevent ATM activation mediated by protein introduced into the cell from the tegument.

To identify the ATM-activating tegument factor, all of the HSV-1 proteins known to be present in the tegument (inner and outer) were screened. Transfection of individual eYFP-tagged tegument protein expression constructs into HEK293 cells failed to identify any single HSV-1 tegument protein as capable of activating ATM (FIG. 24). To address the possibility that more than one tegument protein is necessary for this phenotype, a temperature-sensitive nuclear entry mutant strain, tsB7, was used, which fails to inject the genome after docking to the nuclear pore, yet successfully delivers the entire contents of the tegument into the cell. An absence of pChk2 staining with tsB7 infection was observed at the non-permissive temperature (FIG. 21C), which demonstrated that even the entire tegument is not sufficient to activate ATM, if the viral genome is not delivered to the nucleus.

Taken together, the experiments demonstrate that ATM activation in HSV-1 infection depends on two main factors: 1) the presence of the viral genome in the nucleus, and 2) the availability of an unidentified viral protein that is derived from the tegument and from de novo synthesis. Since both of these components are essential, ATM activation may be achieved as a consequence of an interaction between the viral genome and the responsible protein in the host nucleus.

### ICP0 is Neither Sufficient Nor Necessary for ATM Activation

To gain further insight into the identity of the ATM-activating protein, a synchronized HSV-1 infection in hTCEpi cells was performed, which revealed a surprisingly early onset of ATM activation, with pChk2 staining detectible as early as 20 minutes post infection (FIG. 21D), suggesting immediate early (IE) expression kinetics of the protein in question. Of the six IE proteins of HSV-1, only two are present in the tegument and are known to interact with viral DNA—ICP0 and ICP4.

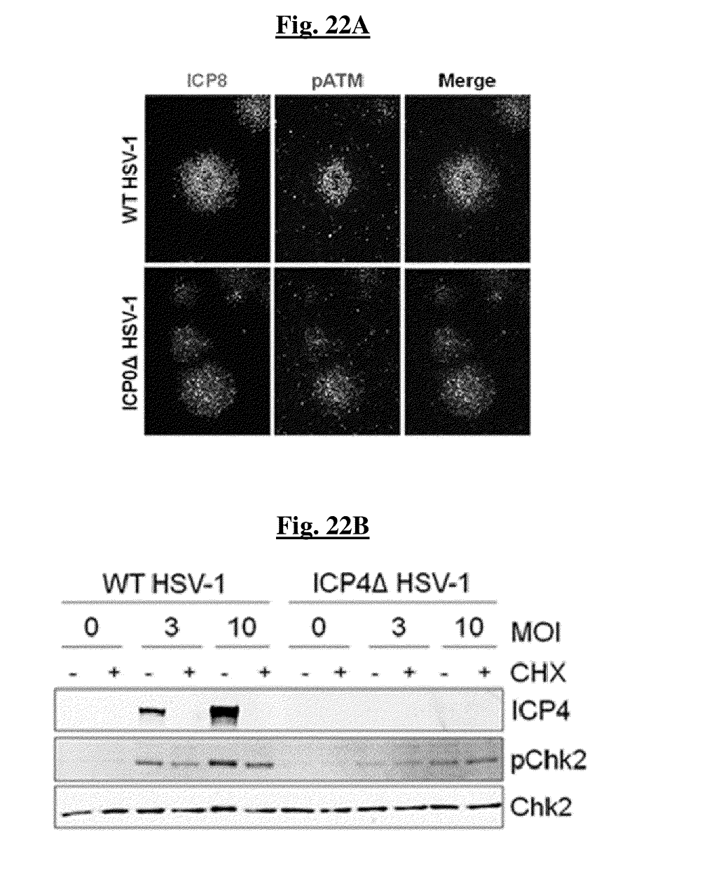

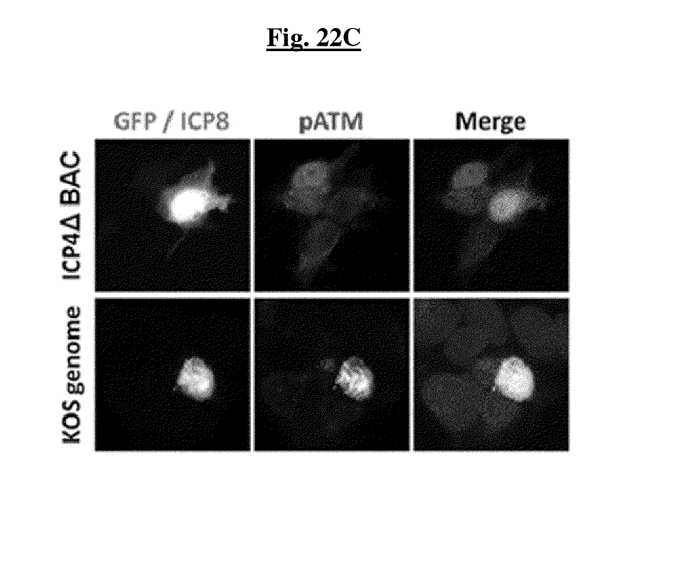

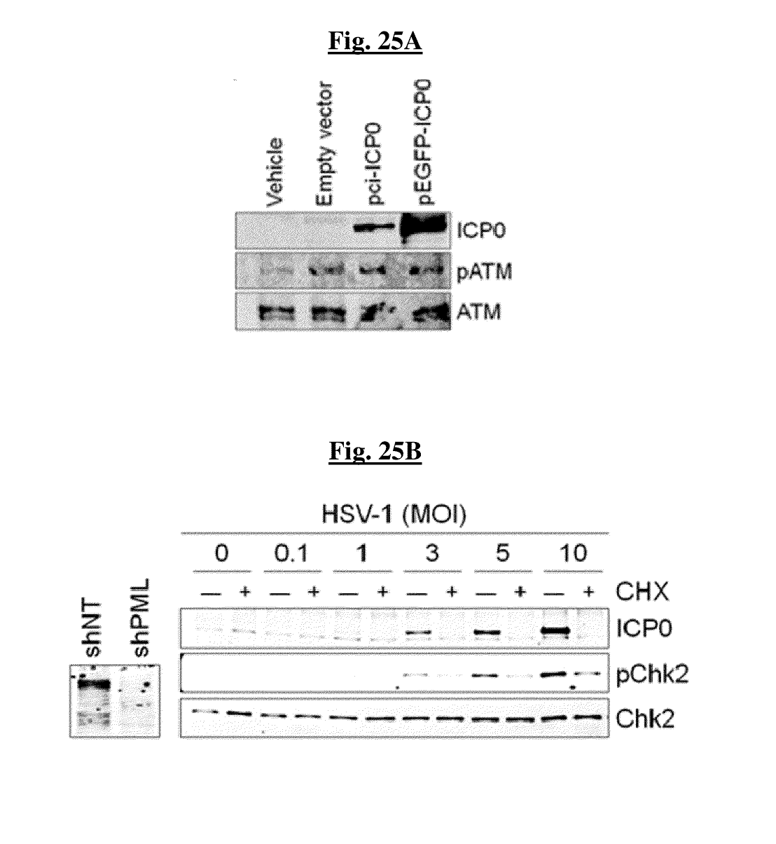

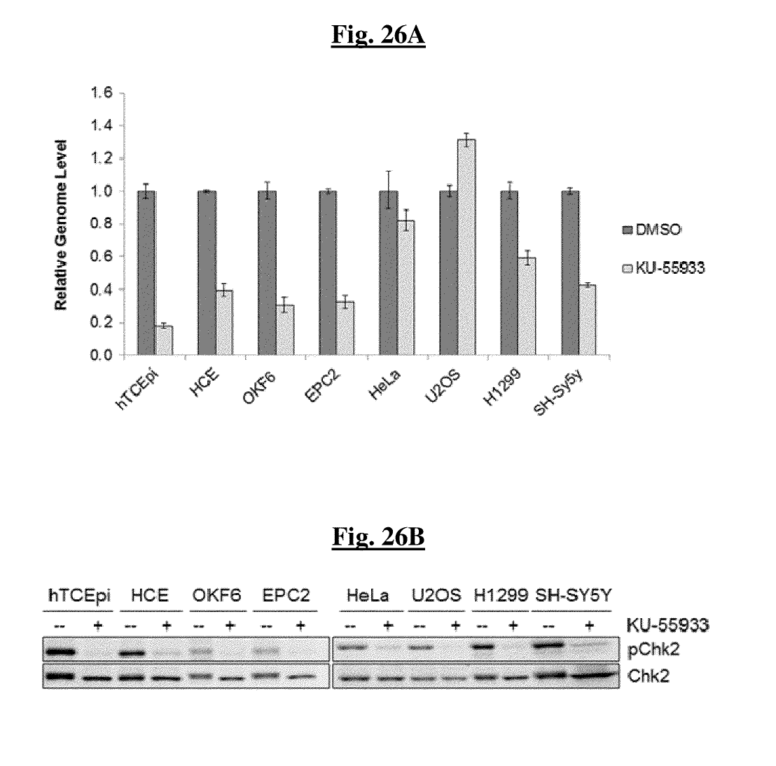

ICP0 interacts with DNA indirectly by influencing the packaging state of the genome through the dispersal of PML bodies, antiviral structures that assemble on the incoming viral genome early during infection. It has not been established whether ICP0 alone is sufficient to activate ATM. In the present study, exogenous expression of ICP0 in HEK293 cells failed to activate ATM, as monitored by immunofluorescence staining (FIG. 24) and by Western blot (FIGS. 25A-25B). Furthermore, ICP0 may be necessary for ATM activation at low MOI but dispensable at high MOI. Since tumorigenic cell lines often have abnormal or dysregulated DDR processes, the role of ICP0 was investigated in a non-tumorigenic cell line, hTCEpi. Interestingly, cells infected with 7134, an ICP0-null strain of HSV-1, or a WT parental strain showed no difference in ATM activation by immunofluorescence (FIG. 22A).

Since PML bodies serve as nuclear depots of numerous DDR proteins, the hypothesis that ICP0 modulates ATM activation through the dispersal of these structures was evaluated. Cycloheximide treatment of PML-depleted hTCEpi cells produced the same partial reduction of pChk2 staining as seen in WT hTCEpi cells (FIG. 25B), indicating that PML bodies do not have a role in ATM activation by HSV-1.

Overall, these findings suggest that ICP0 is neither sufficient nor necessary for HSV-1-induced ATM activation.

### HSV-1 Activates ATM in an ICP4-Dependent Manner

Having eliminated ICP0 as a potential ATM activator, the remaining candidate protein, ICP4, was evaluated. ICP4 has well characterized consensus binding sequences within the viral genome, which it binds as an oligomer or in complex with host proteins. To address the hypothesis that ICP4 is required for ATM activation, pM24, a BAC that contains the full HSV-1 genome with a deletion of both ICP4 coding sequences and constitutively expresses GFP from a CMV promoter, was used. Transfection of this BAC into HEK293 cells failed to produce any detectible ATM activation (FIG. 22C). To confirm this finding, hTCEpi cells were infected with d120, an ICP4-null strain of HSV-1. Compared to WT HSV-1, infection with d120 only achieved the partial level of ATM activation (FIG. 22B). This is consistent with a small amount of ICP4 being present in the tegument, derived from the supporting cells during viral stock production. Importantly, ATM activation by d120 was not affected by CHX, consistent with the hypothesis that the partial inhibition effect is due to the block in de novo ICP4 synthesis.

Taken together, these experiments demonstrate that HSV-1 activates ATM in an ICP4-dependent manner.

### ATM Activity is Critical to HSV-1 Replication Early in the Progress of Infection

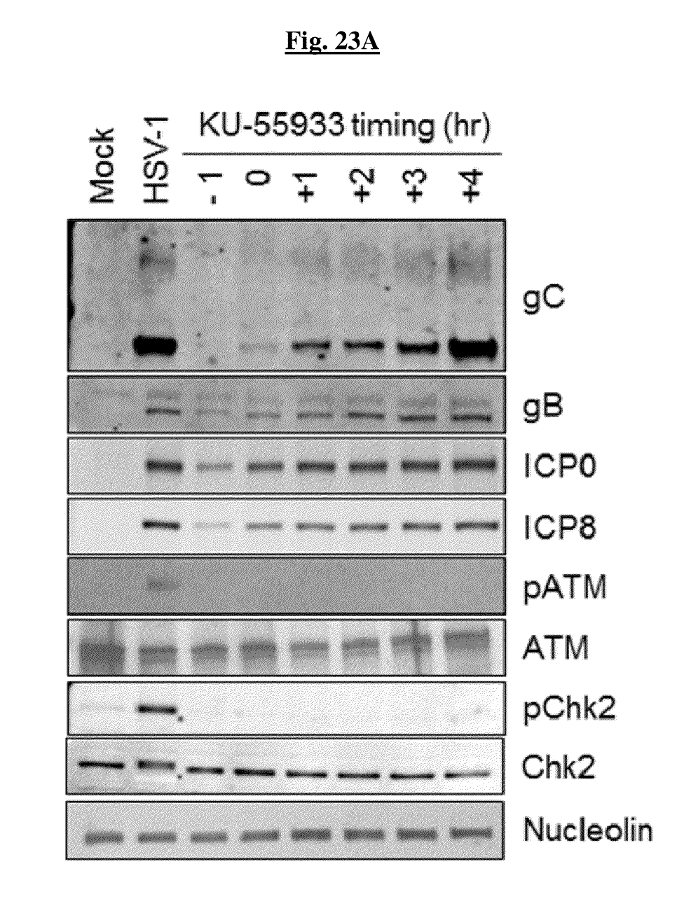

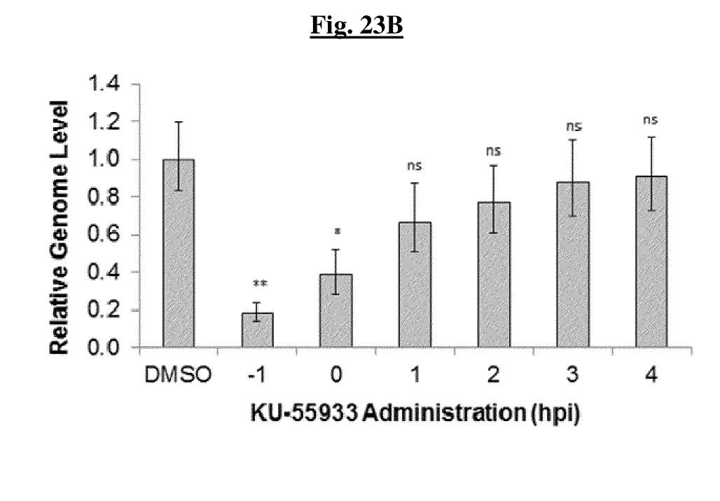

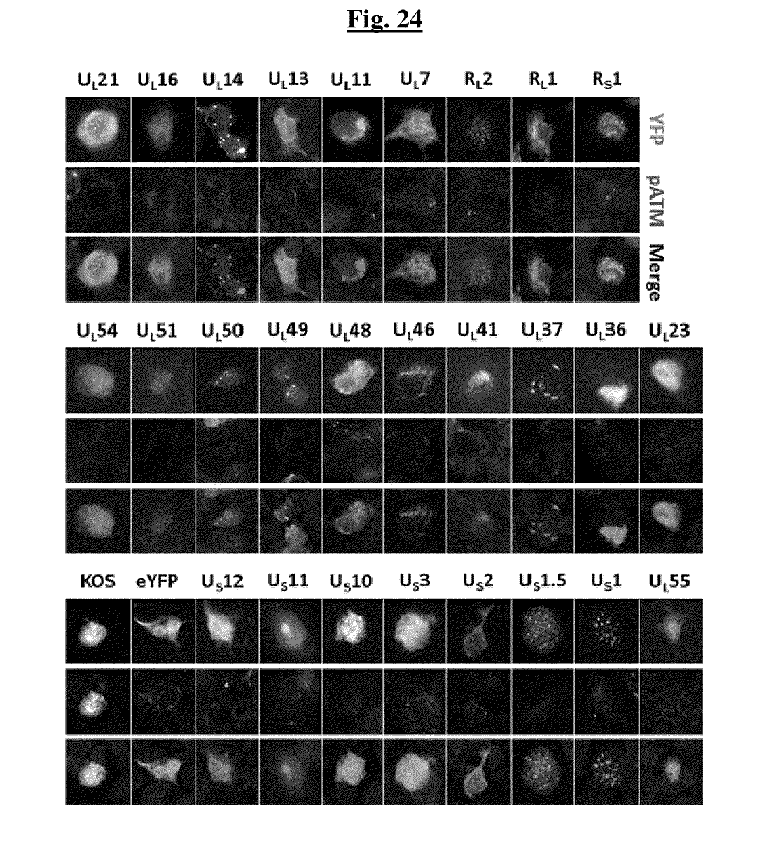

The mechanism whereby ATM activity promotes HSV-1 infection is not known. In order to gain insight into this phenomenon, hTCEpi cells were infected with HSV-1 in the presence of KU-55933, a small molecule inhibitor of ATM. The drug was added to cells at various time points with respect to the start of infection (−1, 0, +1, +2, +3, and +4 hpi), and the experiment was terminated at 8 hpi. Western blot analysis for glycoprotein C (FIG. 23A) and qRT-PCR measurement of viral genome replication (FIG. 23B) showed that KU-55933 treatments prior to the 1 hpi timepoint achieved notable reduction in viral replication, whereas treatments administered at 1 hpi and later had significantly less effect. This result demonstrates the presence of a very early ATM-dependent event in the lytic cycle of HSV-1. Following this event, ATM activity seems to be largely dispensable to the progress of infection.

Taken together, the present studies demonstrate that HSV-1 activates ATM in a manner that is disproportional to the extent of DNA damage incurred by the host during infection, and that the absence of DNA ends and gaps from the viral genome has no effect on its ability to activate ATM. Without wishing to be limited by any theory, these findings provide direct evidence for a non-canonical mechanism of ATM activation, whereby the virus induces rapid and robust DDR activation independently of the presence of DNA lesions.

Identification of the viral factor responsible for ATM activation has presented an experimental challenge. The very early timing of ATM activation observed in the present experiments, along with its independence from viral DNA replication, exclude replication processes as the causative agent for DDR activation. However, it is possible that these structures contribute to sustained ATM activity later during the course of infection, when the nucleus becomes overwhelmed with viral genome copies.

The present studies utilizing exogenous ICP0 expression and ICP0-null virus have shown ICP0 to be neither necessary nor sufficient for the activation of ATM. The present studies utilized normal, highly differentiated, and disease-relevant human epithelial cell lines to provide an accurate model of epithelial infection. In the present experiments, KU-55933 potently suppressed HSV-1 replication in all normal cell types tested (hTCEpi, HCE, OKF6, EPC2), yet had little effect in known transformed or cancer cell lines (HeLa, U2OS, H1299, and SH-SY5Y), which highlights a fundamental difference between normal and cancer cell lines in this context and supports the use of normal cell lines and primary cells for mechanistic investigations of nuclear virus-host interactions (FIGS. 22A-22B).

This study provides strong evidence for the ATM-activating activity of the viral IE protein ICP4. While it is possible that ICP4 activates ATM indirectly via transactivation of another viral factor, the extremely early timing of the activation event and the results of CHX experiments argue against this hypothesis. Without wishing to be limited by any theory, ATM activation at 20 minutes post infection may be achieved only by IE gene products. Since the expression of other IE genes is not upregulated by ICP4, this is unlikely to be a confounding factor in the experiments with the ICP4-null virus.

In certain embodiments, a critical structural or functional ICP4-viral genome interaction takes place in the nucleus. In other embodiments, there is no direct interaction between ICP4 and ATM.

Taken together, the present studies shed light on HSV-1 virus host interactions in epithelial cells. ICP4 orchestrates the viral transcriptional program, activates the host DNA damage response, and breaks down the corneal immune privilege in the context of keratitis—activities that are all critical to the pathogenesis of HSV-1.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the present invention. The appended claims are intended to be construed to include all such embodiments and equivalent variations.

What is claimed is:

1. A composition comprising an anti-herpetic agent and at least one inhibitor selected from the group consisting of an ATM inhibitor, a Chk2 inhibitor, and a salt, solvate or N-oxide thereof, wherein the composition treats or prevents herpes simplex keratitis in a subject in need thereof.

2. The composition of claim 1, wherein the ATM inhibitor is at least one selected from the group consisting of a nucleic acid, siRNA, antisense nucleic acid, ribozyme, peptide, small molecule, antagonist, aptamer, and peptidomimetic.

3. The composition of claim 2, wherein the small molecule is at least one selected from the group consisting of caffeine, wortmannin, chloroquine, CP-466722, KU-55933, KU-59403, KU-60019, and a salt, N-oxide or solvate thereof.

4. The composition of claim 1, wherein the Chk2 inhibitor is at least one selected from the group consisting of a nucleic acid, siRNA, antisense nucleic acid, ribozyme, peptide, small molecule, antagonist, aptamer, and peptidomimetic.

5. The composition of claim 4, wherein the small molecule is at least one selected from the group consisting of Chk2 inhibitor II, SC-203885, NSC-109555, and a salt, N-oxide or solvate thereof.

6. The composition of claim 1, wherein the anti-herpetic agent is at least one selected from the group consisting of acyclovir, famciclovir, penciclovir, valacyclovir, acyclovir, trifluridine, penciclovir and valacyclovir.

7. A method of treating or preventing herpes simplex keratitis in a subject in need thereof, the method comprising administering to the subject an effective amount of an anti-herpetic agent and an effective amount of at least one inhibitor selected from the group consisting of an ATM inhibitor and a Chk2 inhibitor, whereby herpes simplex keratitis is treated or prevented in the subject.

8. The method of claim 7, wherein the ATM inhibitor is at least one selected from the group consisting of a nucleic acid, siRNA, antisense nucleic acid, ribozyme, peptide, small molecule, antagonist, aptamer, and peptidomimetic.

9. The method of claim 8, wherein the small molecule is at least one selected from the group consisting of caffeine, wortmannin, chloroquine, CP-466722, KU-55933, KU-59403, KU-60019, and a salt, N-oxide or solvate thereof.

10. The method of claim 7, wherein the Chk2 inhibitor is selected from the group consisting of a nucleic acid, siRNA, antisense nucleic acid, ribozyme, peptide, small molecule, antagonist, aptamer, and peptidomimetic.

11. The method of claim 10, wherein the small molecule is at least one selected from the group consisting of Chk2 inhibitor II, SC-203885, NSC-109555, and a salt, N-oxide or solvate thereof.

12. The method of claim 7, wherein the anti-herpetic agent is selected from the group consisting of acyclovir, famciclovir, penciclovir, valacyclovir, acyclovir, trifluridine, penciclovir and valacyclovir.

13. The method of claim 7, wherein the at least one inhibitor and the anti-herpetic agent are co-administered to the subject.

14. The method of claim 13, wherein the at least one inhibitor and the anti-herpetic agent are co-formulated.

15. The method of claim 7, wherein the inhibitor is administered to the subject by a topical or intraocular route.

16. The method of claim 7, wherein administration of the inhibitor to the subject reduces the amount of the anti-herpetic agent required to be administered to the subject to obtain the same therapeutic benefit obtained when the effective dose of the anti-herpetic agent in the absence of the inhibitor is administered to the subject.

17. The method of claim 7, wherein the subject experiences less frequent or less severe side effects of the anti-herpetic agent, as compared to when the effective dose of the anti-herpetic agent in the absence of the inhibitor is administered to the subject.

18. The method of claim 7, wherein development of resistance to the anti-herpetic agent is prevented or minimized in the subject, as compared to when the effective dose of the anti-herpetic agent in the absence of the inhibitor is administered to the subject.

19. The method of claim 7, wherein the subject is a mammal.

20. The method of claim 19, wherein the mammal is a human.

21. A method of treating or preventing herpes simplex keratitis in a subject in need thereof, wherein the keratitis is caused by a drug-resistant HSV-1 strain, the method comprising administering to the subject an effective amount of at least one inhibitor selected from the group consisting of an ATM inhibitor and a Chk2 inhibitor, wherein the subject is optionally further administered an effective amount of an anti-herpetic agent, whereby herpes simplex keratitis is treated or prevented in the subject.

22. The method of claim 21, wherein the ATM inhibitor is at least one selected from the group consisting of a nucleic acid, siRNA, antisense nucleic acid, ribozyme, peptide, small molecule, antagonist, aptamer, and peptidomimetic.

23. The method of claim 22, wherein the small molecule is at least one selected from the group consisting of caffeine, wortmannin, chloroquine, CP-466722, KU-55933, KU-59403, KU-60019, and a salt, N-oxide or solvate thereof.

24. The method of claim 21, wherein the Chk2 inhibitor is selected from the group consisting of a nucleic acid, siRNA, antisense nucleic acid, ribozyme, peptide, small molecule, antagonist, aptamer, and peptidomimetic.

25. The method of claim 24, wherein the small molecule is at least one selected from the group consisting of Chk2 inhibitor II, SC-203885, NSC-109555, and a salt, N-oxide or solvate thereof.

26. The method of claim 21, wherein the anti-herpetic agent is at least one selected from the group consisting of acyclovir, famciclovir, penciclovir, valacyclovir, acyclovir, trifluridine, penciclovir and valacyclovir.

27. The method of claim 21, wherein the drug-resistant HSV-1 strain has a TK mutation.

28. The method of claim 21, wherein the strain is resistant to at least one selected from the group consisting of acyclovir, famciclovir, penciclovir, valacyclovir, acyclovir, trifluridine, penciclovir and valacyclovir.

29. The method of claim 21, wherein the subject is a mammal.

30. The method of claim 29, wherein the mammal is a human.

31. A kit comprising at least one inhibitor selected from the group consisting of an ATM inhibitor and a Chk2 inhibitor, the kit further comprising an applicator; and an instructional material for the use of the kit, wherein the instruction material comprises instructions for treating, ameliorating or preventing herpes simplex keratitis in a subject in need thereof.

32. The kit of claim 30, wherein the kit further comprises an anti-herpetic agent.
